(19) United States
(12) Patent Application Publication
(10) Pub. No.: US 2025/0360207 A1
(43) Pub. Date: Nov. 27, 2025

(54) METHODS FOR TREATING LUPUS NEPHRITIS USING FCRN ANTAGONISTS

(71) Applicant: argenx BV, Ghent (BE)

(72) Inventors: Tim Van Hauwermeiren, Ghent (BE); Peter Verheesen, Ghent (BE); Magdalena Sips, Ghent (BE)

(21) Appl. No.: 19/200,764

(22) Filed: May 7, 2025

Related U.S. Application Data
(63) Continuation of application No. PCT/IB2023/000679, filed on Nov. 7, 2023.
(60) Provisional application No. 63/382,566, filed on Nov. 7, 2022.

(51) Int. Cl. A61K 39/395; A61K 31/365; A61K 31/4706; A61K 31/573; A61K 39/00; A61K 45/06; C07K 16/28

(52) U.S. Cl. CPC A61K 39/39541; A61K 31/365; A61K 31/4706; A61K 31/573; A61K 45/06; C07K 16/283; A61K 2039/54; A61K 2039/545; C07K 2317/52

(57) ABSTRACT

Provided herein are methods of treating lupus nephritis using an effective amount of a human neonatal Fc receptor (FcRn) antagonist. FcRn antagonists for use in the treatment of lupus nephritis and for use in the manufacture of a medicament for the treatment of lupus nephritis are also provided herein.

### RELATED APPLICATIONS

This application is a Continuation of International Patent Application No. PCT/IB2023/000679, filed Nov. 7, 2023, which claims priority to U.S. Provisional Patent Application Ser. No. 63/382,566, filed Nov. 7, 2022, the contents of each of which are incorporated herein by reference in their entirety.

### REFERENCE TO SEQUENCE LISTING

This application contains a sequence listing which has been submitted electronically in ST.26 format and is hereby incorporated by reference in its entirety (said ST.26 copy, created on Nov. 6, 2023, is named “205806_SL.xml” and is 40,649 bytes in size).

### FIELD

The present disclosure relates to methods of treating lupus nephritis (LN). The methods involve use of an antagonist of human neonatal Fc receptor (FcRn), which in certain embodiments is efgartigimod.

### BACKGROUND

It is estimated that more than 2.5% of the human population is affected by autoantibody-driven autoimmune diseases, in which autoreactive antibodies are directly pathogenic. Systemic lupus erythematosus (SLE) is a chronic heterogenous autoimmune disease. LN is an inflammatory autoimmune disease of the kidney caused by SLE and is the most common life-threatening manifestation of SLE.

In unselected patients with SLE, approximately 25% to 50% have signs or symptoms of kidney disease at SLE onset, and approximately 40% to 60% of patients with SLE will develop renal involvement during the course of the disease. In China, approximately half of the patients with SLE have renal involvement.

In active proliferative LN, induction treatment with mycophenolate mofetil (MMF) or mycophenolic acid (MPA), or low-dose intravenous (IV) cyclophosphamide (CYC), both combined with glucocorticoids, is recommended. Alternative regimens include treatment with MMF and calcineurin inhibitors (e.g., tacrolimus), and high-dose CYC. Subsequent maintenance therapy includes treatment with MMF or azathioprine with no or low-dose glucocorticoids. Despite aggressive immunosuppressive therapy, among patients with LN, 10% to 30% of patients still progress to end-stage renal disease (ESRD), the ultimate manifestation of LN. Furthermore, up to 60% of the patients are unable to achieve the treatment targets with currently available therapeutic options.

Accordingly, there is a need in the art for improved LN treatment options.

Therapeutic antagonism of FcRn, a major histocompatibility complex class I-like molecule that is involved in the recycling of immunoglobulin G (IgG) and is thus responsible for the long half-life of IgG, has been explored as a strategy to treat IgG-mediated autoimmune diseases such as generalized myasthenia gravis (gMG), immune thrombocytopenia (ITP), and pemphigus (pemphigus vulgaris (PV) and pemphigus foliaceus (PF)). The remarkable clinical efficacy of FcRn antagonism appears to be directly linked to early removal of pathogenic IgG autoantibodies from circulation.

Pathogenic autoantibodies and complement deposits are critically involved in SLE pathogenesis, particularly LN, where renal deposition of immune complexes is a hallmark of the disease. By reducing accumulated pathogenic autoantibodies in renal glomeruli, FcRn antagonists may provide a safer, more effective treatment option for patients with LN.

### SUMMARY

The present disclosure is broadly directed to methods for treating lupus nephritis with FcRn antagonists.

The present disclosure provides a method of treating lupus nephritis in a subject in need thereof, the method comprising administering to the subject an effective amount of a human neonatal Fc receptor (FcRn) antagonist. In some embodiments, the FcRn antagonist comprises two, three, or four FcRn binding regions. In some embodiments, the FcRn antagonist comprises or consists of a variant Fc region or FcRn binding fragment thereof. In some embodiments, the variant Fc region or FcRn binding fragment thereof binds to FcRn with a higher affinity at pH 6.0 as compared to a corresponding wild-type Fc region. In some embodiments, the variant Fc region or FcRn binding fragment thereof binds to FcRn with a higher affinity at pH 7.4 as compared to a corresponding wild-type Fc region.

In some embodiments, the variant Fc region comprises or consists of a first Fc domain and a second Fc domain which form a homodimer or heterodimer. In some embodiments, the first Fc domain and/or the second Fc domain comprise amino acids Y, T, E, K, and F at EU positions 252, 254, 256, 433, and 434, respectively. In some embodiments, the first Fc domain and/or the second Fc domain comprise amino acids Y, T, E, K, F, and Y at EU positions 252, 254, 256, 433, 434, and 436, respectively.

In some embodiments, the first Fc domain and/or the second Fc domain comprise an amino acid sequence independently selected from the group consisting of SEQ ID NO: 1, SEQ ID NO: 2, SEQ ID NO: 3, and SEQ ID NO: 30. In some embodiments, the first Fc domain and the second Fc domain comprise an amino acid sequence independently selected from the group consisting of SEQ ID NO: 1, SEQ ID NO: 2, SEQ ID NO: 3, and SEQ ID NO: 30. In some embodiments, the FcRn antagonist is efgartigimod.

In some embodiments, the FcRn antagonist is an anti-FcRn antibody.

In some embodiments, the FcRn antagonist is administered to the subject at a fixed dose of 20 mg to 20,000 mg or at a dose of 0.2 mg/kg to 200 mg/kg. In some embodiments, the FcRn antagonist is administered intravenously at a dose of 10 mg/kg to 30 mg/kg once weekly or once every two weeks. In some embodiments, the FcRn antagonist is administered intravenously at a dose of 10 mg/kg once weekly. In some embodiments, the FcRn antagonist is administered subcutaneously at a fixed dose of 750 mg to 3000 mg once weekly, once every two weeks, once every three weeks, once every four weeks, or once monthly. In some embodiments, the FcRn antagonist is administered subcutaneously at a fixed dose of 1000 mg or 2000 mg once weekly or once every two weeks.

In some embodiments, the FcRn antagonist is administered for 52 weeks or less. In some embodiments, the FcRn antagonist is administered for 24 weeks or less. In some embodiments, the FcRn antagonist is administered for 24 weeks. In some embodiments, the FcRn antagonist is administered for at least 24 weeks. In some embodiments, the FcRn antagonist is administered for at least 52 weeks.

In some embodiments, the method also includes administering to the subject an effective amount of a glucocorticoid, mycophenolate mofetil (MMF), mycophenolic acid (MPA), an angiotensin-converting enzyme inhibitor (ACEi), an angiotensin receptor blocker (ARB), hydroxychloroquine, a B-lymphocyte targeting agent, or any combination thereof.

In some embodiments, the glucocorticoid is methylprednisolone. In some embodiments, the methylprednisolone is administered intravenously at a dose of 250 mg to 500 mg once a day. In some embodiments, the methylprednisolone is administered daily for 1 to 3 days. In some embodiments, the methylprednisolone is administered for 3 days.

In some embodiments, the glucocorticoid is prednisone. In some embodiments, the prednisone is administered orally at a dose of 10 mg/day to 60 mg/day. In some embodiments, the prednisone is administered orally at a dose of 0.5 mg/kg/day to 1 mg/kg/day. In some embodiments, when the subject is administered the FcRn antagonist, the dose of prednisone is tapered over 12 weeks to a dose of 7.5 mg/day.

In some embodiments, the MMF or MPA is administered daily. In some embodiments, the MMF or MPA is administered at a daily dose, wherein the daily dose is up-titrated to 1.5 g/day to 2 g/day in divided doses within 4 weeks.

In some embodiments, the hydroxychloroquine is administered at a dose of up to 5 mg/kg/day. In some embodiments, the hydroxychloroquine is administered for 24 weeks. In some embodiments, the hydroxychloroquine is administered for at least 24 weeks.

In some embodiments, the B-lymphocyte targeting agent is selected from the group consisting of belimumab, rituximab, and obinutuzumab.

The present disclosure also provides a method of treating lupus nephritis in a subject in need thereof, the method comprising administering to the subject an effective amount of an FcRn antagonist within 60 days of the subject receiving an induction therapy for lupus nephritis. In some embodiments, the induction therapy comprises administering to the subject an effective amount of a glucocorticoid.

In some embodiments, the induction therapy comprises administering to the subject an effective amount of methylprednisolone. In some embodiments, the methylprednisolone is administered intravenously at a dose of 250 mg to 500 mg once a day. In some embodiments, the methylprednisolone is administered daily for 1 to 3 days. In some embodiments, the methylprednisolone is administered for 3 days.

In some embodiments, the induction therapy comprises administering prednisone to the subject. In some embodiments, the prednisone is administered orally at a dose of 10 mg/day to 60 mg/day. In some embodiments, the prednisone is administered orally at a dose of 0.5 mg/kg/day to 1 mg/kg/day. In some embodiments, when the subject is administered the FcRn antagonist, the dose of prednisone is tapered over 12 weeks to a dose of 7.5 mg/day.

In some embodiments, the induction therapy further comprises administering to the subject a daily dose of an effective amount of MMF or MPA. In some embodiments, the daily dose of MMF or MPA is up-titrated to 1.5 g/day to 2 g/day in divided doses within 4 weeks.

In some embodiments, the induction therapy also includes administering to the subject a dose of an effective amount of hydroxychloroquine. In some embodiments, the dose of hydroxychloroquine is up to 5 mg/kg/day. In some embodiments, the hydroxychloroquine is administered for 24 weeks. In some embodiments, the dose of hydroxychloroquine is administered for at least 24 weeks.

In some embodiments, the induction therapy also includes administering to the subject an effective amount of a B-lymphocyte targeting agent. In some embodiments, the B-lymphocyte targeting agent is selected from the group consisting of belimumab, rituximab, and obinutuzumab.

In some embodiments, after administering the FcRn antagonist to the subject, the subject exhibits a post-administration estimated glomerular filtration rate (eGFR) of at least 60 mL/min/1.73 m². In some embodiments, after administering the FcRn antagonist to the subject, the subject exhibits a post-administration eGFR that is less than 20% reduced as compared to a baseline eGFR obtained from the subject prior to administering the FcRn antagonist. In some embodiments, the post-administration eGFR is measured 24 or 52 weeks after administering the FcRn antagonist to the subject. In some embodiments, after administering the FcRn antagonist to the subject, the subject exhibits a post-administration eGFR of at least 90 mL/min/1.73 m². In some embodiments, after administering the FcRn antagonist to the subject, the subject exhibits a post-administration eGFR that is less than 10% reduced as compared to a baseline eGFR obtained from the subject prior to administering the FcRn antagonist. In some embodiments, the post-administration eGFR is measured 24 or 52 weeks after administering the FcRn antagonist to the subject.

In some embodiments, after administering the FcRn antagonist to the subject, the subject exhibits a post-administration urine protein creatinine ratio (UPCR) of at most 0.5 mg/mg. In some embodiments, after administering the FcRn antagonist to the subject, the subject exhibits a post-administration UPCR that is at least 50% reduced as compared to a baseline UPCR obtained from the subject prior to administering the FcRn antagonist, and wherein if the baseline UPCR is at most 3 mg/mg, then the post-administration UPCR is less than 1 mg/mg. In some embodiments, after administering the FcRn antagonist to the subject, the subject exhibits a post-administration UPCR that is at least 50% reduced as compared to a baseline UPCR obtained from the subject prior to administering the FcRn antagonist, and wherein if the baseline UPCR is greater than 3 mg/mg, then the post-administration UPCR is less than 3 mg/mg. In some embodiments, the post-administration UPCR is measured 24 or 52 weeks after administering the FcRn antagonist to the subject.

In some embodiments, after administering the FcRn antagonist to the subject, the subject exhibits a post-administration Systemic Lupus Erythematosus Disease Activity Index (SLEDAI)-2K score that is reduced as compared to a baseline SLEDAI-2K score obtained from the subject prior to administering the FcRn antagonist. In some embodiments, the post-administration SLEDAI-2K score is measured 24 or 52 weeks after administering the FcRn antagonist to the subject.

In some embodiments, after administering the FcRn antagonist to the subject, the subject exhibits a post-administration level of a serum autoantibody that is reduced as compared to a baseline level of the serum autoantibody obtained from the subject prior to administering the FcRn antagonist. In some embodiments, the post-administration level of the serum autoantibody is reduced by at least 10%, 20%, 30%, 40%, 50%, 60%, 70%, 80%, 90%, or 100%, as compared to the baseline level of the serum autoantibody obtained from the subject prior to administering the FcRn antagonist. In some embodiments, the post-administration level of the serum autoantibody is measured 24 or 52 weeks after administering the FcRn antagonist to the subject. In some embodiments, the serum autoantibody is selected from the group consisting of anti-dsDNA, ANA, aCL, anti-Sm, and anti-C1q.

In some embodiments, after administering the FcRn antagonist to the subject, the subject exhibits a post-administration level of a serum complement that is reduced as compared to a baseline level of the serum complement obtained from the subject prior to administering the FcRn antagonist. In some embodiments, the post-administration level of the serum complement is reduced by at least 10%, 20%, 30%, 40%, 50%, 60%, 70%, 80%, 90%, or 100%, as compared to the baseline level of the serum complement obtained from the subject prior to administering the FcRn antagonist. In some embodiments, the post-administration level of the serum complement is measured 24 or 52 weeks after administering the FcRn antagonist to the subject. In some embodiments, the serum complement is selected from the group consisting of C3, C4, CH50, and C1q-binding circulating immune complexes.

In some embodiments, after administering the FcRn antagonist to the subject, the subject exhibits a post-administration level of serum IgG that is reduced as compared to a baseline level of serum IgG obtained from the subject prior to administering the FcRn antagonist. In some embodiments, the post-administration level of serum IgG is reduced by at least 10%, 20%, 30%, 40%, 50%, 60%, 70%, 80%, 90%, or 100%, as compared to the baseline level of serum IgG obtained from the subject prior to administering the FcRn antagonist. In some embodiments, the post-administration level of serum IgG is measured 24 or 52 weeks after administering the FcRn antagonist to the subject.

In some embodiments, after administering the FcRn antagonist to the subject, the subject exhibits a post-administration level of serum albumin that is not reduced as compared to a baseline level of serum albumin obtained from the subject prior to administering the FcRn antagonist.

In some embodiments, the FcRn antagonist is in an aqueous solution comprising about 4 mM sodium phosphate, about 146 mM sodium chloride, about 24 mM L-arginine, and about 0.0032% (w/v) polysorbate 80, wherein the composition has a pH of about 6.7. In some embodiments, the aqueous solution comprises about 3.2 mg/ml of the FcRn antagonist.

In some embodiments, the FcRn antagonist is in an aqueous solution comprising about 20 mM L-histidine, about 100 mM sodium chloride, about 60 mM sucrose, about 10 mM L-methionine, and about 0.04% (w/v) polysorbate 20, wherein the composition has a pH of about 6.0. In some embodiments, the aqueous solution comprises about 180 mg/ml of the FcRn antagonist.

In some embodiments, the FcRn antagonist is in an aqueous solution comprising about 20 mM L-histidine, about 50 mM L-arginine, about 100 mM sodium chloride, about 60 mM sucrose, about 10 mM L-methionine, and about 0.04% (w/v) polysorbate 80, wherein the composition has a pH of about 6.0. In some embodiments, the aqueous solution comprises about 200 mg/ml of the FcRn antagonist.

The present disclosure also provides an FcRn antagonist for use in the treatment of lupus nephritis, wherein the treatment is performed according to the methods described above and herein.

The present disclosure also provides an FcRn antagonist for use in the manufacture of a medicament for the treatment of lupus nephritis, wherein the treatment is performed according to the methods described above and herein.

The present disclosure also provides a use of an FcRn antagonist for the treatment of lupus nephritis according to the methods described above and herein.

The present disclosure also provides a use of an FcRn antagonist for the manufacture of a medicament for treatment of lupus nephritis, wherein the treatment is performed according to the methods described above and herein.

### BRIEF DESCRIPTION OF THE DRAWINGS

FIG. 1 is a set of graphs showing levels of IgG circulating immune complexes (IgG CIC) levels (top panels) and disease severity (bottom panels) in human subjects with pemphigus vulgaris (PV; n=6) and pemphigus foliaceus (PF; n=6) following efgartigimod treatment. Black lines indicate median. Dotted line indicates CS (clinical significance). PDAI: pemphigus disease area index; CR: complete clinical remission; EoT: end of treatment; EoS: end of study. The data demonstrate that efgartigimod reduced levels of circulating immune complexes.

### DETAILED DESCRIPTION

The present disclosure provides engineered FcRn antagonists and methods for their use in treating LN. Advantageously, the methods disclosed herein permit long-term preservation of kidney function, prevention of disease flares, prevention of organ damage, management of comorbidities, improvement in patient survival, and improvement in disease-related quality of life.

### Definitions

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “FcRn antagonist” refers to any agent that binds specifically to FcRn and inhibits the binding of immunoglobulin to FcRn (e.g., human FcRn). In an embodiment, the FcRn antagonist is an Fc region (e.g., a variant Fc region disclosed herein) that specifically binds to FcRn through the Fc region and inhibits the binding of immunoglobulin to FcRn. In an embodiment, the FcRn antagonist is not a full-length IgG antibody. In an embodiment, the FcRn antagonist comprises an antigen binding site that binds a target antigen and a variant Fc region. In an embodiment, the FcRn antagonist is an Fc fragment comprising or consisting of an Fc region and lacking an antigen binding site. In an embodiment, the term “FcRn antagonist” refers to an antibody or antigen-binding fragment thereof that specifically binds to FcRn via its antigen binding domain or via its Fc region and inhibits the binding of the Fc region of immunoglobulin (e.g., IgG autoantibodies) to FcRn.

As used herein, the terms “antibody” and “antibodies” include full-length antibodies, antigen-binding fragments of full-length antibodies, and molecules comprising antibody CDRs, VH regions, or VL regions. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies (sdAb), monovalent antibodies, single chain antibodies or single-chain Fvs (scFv), camelid antibodies, affibody molecules, humanized antibodies, VHH fragments, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. Antibodies can be of any type (e.g., IgG, IgE, IgM, IgD, IgA, or IgY), any class (e.g., IgG1, IgG₂, IgG₃, IgG₄, IgA₁ or IgA₂), or any subclass (e.g., IgG₂ₐ or IgG_{2b}) of immunoglobulin molecule.

As used herein, the term “Fc domain” refers to the portion of a single immunoglobulin heavy chain comprising both the CH2 and CH3 domains of the antibody. In some embodiments, the Fc domain comprises at least a portion of a hinge (e.g., upper, middle, and/or lower hinge region) region, a CH2 domain, and a CH3 domain. In some embodiments, the Fc domain does not include the hinge region.

As used herein, the term “hinge region” refers to the portion of a heavy chain molecule that joins the CH1 domain to the CH2 domain. In some embodiments, the hinge region is at most 70 amino acid residues in length. In some embodiments, the hinge region comprises approximately 25 amino acid residues and is flexible, thus allowing the two N-terminal antigen binding regions to move independently. In some embodiments, this hinge region comprises approximately 11-17 amino acid residues and is flexible, thus allowing the two N-terminal antigen binding regions to move independently. In some embodiments, the hinge region is 12 amino acid residues in length. In some embodiments, the hinge region is 15 amino acid residues in length. In some embodiments, the hinge region is 62 amino acid residues in length. Hinge regions can be subdivided into three distinct domains: upper, middle, and lower hinge domains. The FcRn antagonists of the instant disclosure can include all or any portion of a hinge region. In some embodiments, the hinge region is from an IgG1 antibody. In some embodiments, the hinge region comprises the amino acid sequence of EPKSCDKTHTCPPCP (SEQ ID NO: 12).

As used herein, the term “Fc region” refers to the portion of an immunoglobulin formed by the Fc domains of its two heavy chains. The Fc region can be a wild-type Fc region (native Fc region) or a variant Fc region. A native Fc region is homodimeric. The Fc region can be derived from any native immunoglobulin. In some embodiments, the is formed from an IgA, IgD, IgE, or IgG heavy chain constant region. In some embodiments, the Fc region is formed from an IgG heavy chain constant region. In some embodiments, the IgG heavy chain is an IgG1, IgG2, IgG3 or IgG4 heavy chain constant region. In some embodiments, the Fc region is formed from an IgG1 heavy chain constant region. In some embodiments, the IgG1 heavy chain constant region comprises a G1m1(a), G1m2(x), G1m3(f), or G1m17(z) allotype. See, e.g., Jefferis and Lefranc (2009) mAbs 1(4): 332-338, and de Taeye et al. (2020) Front Immunol. 11:740, incorporated herein by reference in their entirety.

As used herein, the term “variant Fc region” refers to an Fc region with one or more alteration(s) relative to a native Fc region. Alterations can include amino acid substitutions, additions and/or deletions, linkage of additional moieties, and/or alteration of the native glycans. The term encompasses heterodimeric Fc regions where each of the constituent Fc domains is different. The term also encompasses single chain Fc regions where the constituent Fc domains are linked together by a linker moiety.

As used herein the term “FcRn binding fragment” refers to a portion of an Fc region that is sufficient to confer FcRn binding.

As used herein, the term “EU position” refers to the amino acid position in the EU numbering convention for the Fc region described in Edelman, G M et al., Proc. Natl. Acad. USA, 63, 78-85 (1969) and Rabat et al., in “Sequences of Proteins of Immunological Interest,” U.S. Dept. Health and Human Services, 5th edition, 1991.

As used herein, the term “baseline” refers to a measurement (e.g., a frequency of B cells, IgG levels) in a patient, e.g., in a patient's blood or urine, prior to the first administration (e.g., intravenous, or subcutaneous administration) of a treatment (e.g., an FcRn antagonist).

As used herein, the term “autoantibody-mediated disease” refers to any disease or disorder in which the underlying pathology is caused, at least in part, by pathogenic IgG autoantibodies.

As used herein, the term “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “dose” or “dosing” refers to an amount of an agent administered to a subject in a single administration.

As used herein, the terms “fixed dose” or “flat dose” both refer to a dose that does not vary based upon a characteristic (e.g., body mass, e.g., within a set range; sex; age, e.g., within a set range; etc.) of the subject.

As used herein, the term “remission” refers to a patient who has no new markers of an autoantibody-mediated disease and the baseline markers of the disease have completely resolved or healed.

As used herein, the term “relapse” or “flare” refers to a patient with an autoantibody-mediated disease who has an appearance of physical symptoms and/or an increase of a marker of the autoantibody-mediated disease after a period of remission of the autoantibody-mediated disease. In some embodiments, the flare is an LN flare. In some embodiments, the LN flare is characterized by a 20% increase of urine protein creatinine ratio (UPCR) from baseline. In some embodiments, the flare is an extrarenal SLE flare. In some embodiments, the extrarenal SLE flare is characterized by an increase in the SLEDAI-2K score that is not accounted for by either hypocomplementemia, an increase in anti-dsDNA antibody level, the initiation of new immunosuppressive therapy, or an increase of corticosteroids.

As used herein, the term “subject” or “patient” or “participant” includes any human or non-human animal. In an embodiment, the subject or patient or participant is a human or non-human mammal. In an embodiment, the subject or patient or participant is a human. In some embodiments, the human subject or patient or participant is of Asian descent.

As used herein, the term “about” or “approximately” when referring to a measurable value, such as a dosage, encompasses variations of 20%, ±15%, ±10%, ±5%, ±1%, or ±0.10% of a given value or range, as are appropriate to perform the methods disclosed herein.

### Lupus Nephritis

Systemic lupus erythematosus (SLE) is a chronic disease, complicated and challenging to diagnose and treat. It is a heterogenous autoimmune disease characterized by the dysregulation of T and B lineage cells and other components of the innate immune system. A hallmark of the disease is the production of pathogenic autoantibodies against double-stranded DNA (dsDNA), phospholipids, blood cells, and other targets.

SLE manifestations are associated with multiple autoantibodies, ensuing immune complex formation and deposition, and other immune processes. LN, an inflammatory autoimmune disease of the kidney caused by SLE, is the most common life-threatening manifestation of systemic lupus. It is primarily caused by a type-3, hypersensitivity reaction, in which anti-double-stranded DNA (anti-dsDNA) binds to DNA, which forms an anti-dsDNA immune complex. These immune complexes deposit on the mesangium, subendothelial, and/or subepithelial space near the glomerular basement membrane of the kidney. This leads to an inflammatory response with the onset of LN, in which the complement pathway is activated with a resultant influx of neutrophils and other inflammatory cells. Approximately 40% to 60% of patients with SLE will develop renal involvement at any time during the course of SLE disease, with substantial morbidity or mortality. Approximately 25-50% of patients with SLE already have signs or symptoms of kidney disease at SLE diagnosis. In China, approximately half of the patients with SLE have renal involvement.

All patients with LN are considered to have chronic kidney disease (CKD), which progresses to end-stage renal disease (ESRD) in part of the patients with LN. In China, LN is one of the most common causes of ESRD. Symptoms of LN include blood in the urine, foamy urine, proteinuria, high blood pressure, edema, joint pain, muscle pain, malar rash, and high levels of creatine in the blood.

LN is histologically classified into 6 distinct classes that represent different manifestations and severities of renal involvement in SLE. The rationale for classifying LN into different classes has also taken the differences in prognosis into consideration. Glomerulonephritis is the most common form of renal disease in SLE, but it is frequently accompanied by tubulointerstitial and vascular lesions. In general, LN with Class VI represents kidney atrophy in ESRD. Patients with proliferative LN of varying degrees (Class III, IV, or V), but not Class I or II LN, are at immediate risk of CKD progression. These classes are the most severe manifestations of renal pathology, and they require aggressive immunosuppressive treatment to prevent progression to ESRD and to minimize the associated morbidity and mortality.

The goals for treatment include patient survival, long-term preservation of kidney function, prevention of disease flares, prevention of organ damage, management of comorbidities, and improvement in disease-related quality of life. In European League Against Rheumatism and European Renal Association-European Dialysis and Transplant Association (EULAR/ERA-EDTA) recommendations, in active proliferative LN, initial (induction) treatment with mycophenolate mofetil (MMF, 2 g to 3 g/day or mycophenolic acid [MPA] at equivalent dose) or low-dose intravenous (IV) cyclophosphamide (CYC; 500 mg×6 biweekly doses), both combined with glucocorticoids (pulses of IV methylprednisolone, then oral prednisone) is recommended. MMF/calcineurin inhibitors (CNIs) (especially tacrolimus) combination and high dose CYC are alternatives, for patients with nephrotic range proteinuria and adverse prognostic factors. Subsequent long-term maintenance treatment with MMF or azathioprine should follow, with no or low dose (<7.5 mg/day) glucocorticoids. Similarly, in the Kidney Disease: Improving Global Outcomes (KDIGO) clinical practice guidelines, for patients with active LN, initial treatment includes corticosteroids (IV methylprednisolone 0.25-0.5 g/day for 1 to 3 days, then oral prednisone 0.6-1 mg/kg/day [not to exceed 80 mg/day] and tapered over a few months to maintenance dosage), and calcineurin inhibitors (e.g., tacrolimus and voclosporin), mycophenolic acid analogs (e.g., MMF or mycophenolic acid sodium), and CYC. See, KDIGO 2021 Clinical Practice Guideline for the Management of Glomerular Diseases, Kidney Intl. (2021) 100 (45): 51-S276, which is incorporated herein by reference in its entirety. In addition, KDIGO recommends initial treatment of B-lymphocyte targeting biologics, for example, without limitation, belimumab and rituximab.

Despite aggressive immunosuppressive therapy, among patients with LN, 10% to 30% of patients still progress to ESRD. Whereas these outcomes have improved over the past 30 years, continued risk of treatment failure remains (as defined by death, ESRD, sustained doubling of serum creatinine, LN flare, or need for rescue medications). There is a clear need for more effective treatments, because up to 60% of the patients are unable to achieve the treatment targets with current therapeutic options.

Substantial evidence has shown that an early reduction in proteinuria, particularly within 6 to 12 months from the start of treatment, is the single best predictor of improved long-term outcomes, including reduced risk of disease flares, ESRD, and death. As such, proteinuria, or UPCR, have been useful parameters to evaluate the treatment effect and are preferred outcome measures by the regulatory agencies. Early reduction in UPCR indicates a long-term benefit in treatment of LN.

### FcRn Antagonists

FcRn antagonists that are useful in the methods and uses provided herein include any molecule that binds to and inhibits FcRn, including, but not limited to, any anti-FcRn antibody, any anti-FcRn binding region, or any Fc domain or Fc region.

In some embodiments, the FcRn antagonists disclosed herein comprise two, three, or four FcRn binding regions, such as an Fc region.

Any Fc region can be altered to produce a variant Fc region for use in the methods disclosed herein. In general, an Fc region, or FcRn-binding fragment thereof, is from a human immunoglobulin. It is understood, however, that the Fc region may be derived from an immunoglobulin of any other mammalian species, including for example, a camelid species, a rodent (e.g., a mouse, rat, rabbit, guinea pig) or non-human primate (e.g., chimpanzee, macaque) species. Moreover, the Fc region or portion thereof may be derived from any immunoglobulin class, including IgM, IgG, IgD, IgA, and IgE, and any immunoglobulin isotype, including IgG1, IgG2, IgG3, and IgG4. In an embodiment, the Fc region is an IgG Fc region (e.g., a human IgG region). In an embodiment, the Fc region is an IgG1 Fc region (e.g., a human IgG1 region). In an embodiment, the Fc region is a chimeric Fc region comprising portions of several different Fc regions. Suitable examples of chimeric Fc regions are set forth in US 2011/0243966A1, which is incorporated herein by reference in its entirety. A variety of Fc region gene sequences (e.g., human constant region gene sequences) are available in the form of publicly accessible deposits.

An Fc region can be further truncated or internally deleted to produce a minimal FcRn-binding fragment thereof. The ability of an Fc-region fragment to bind to FcRn can be determined using any art recognized binding assay (e.g., ELISA).

To enhance the manufacturability of the FcRn antagonists disclosed herein, it is preferable that the constituent Fc regions do not comprise any non-disulfide bonded cysteine residues. Accordingly, in an embodiment, the Fc regions do not comprise a free cysteine residue.

Any Fc variant, or FcRn-binding fragment thereof, that binds specifically to FcRn with increased affinity and reduced pH dependence relative to the native (i.e., wild-type) Fc region can be used in the methods disclosed herein. In an embodiment, the variant Fc region comprises amino acid alterations, substitutions, insertions and/or deletions that confer the desired characteristics. In some embodiments, the FcRn antagonist comprises a variant Fc region, or FcRn binding fragment thereof, which binds to FcRn with a higher affinity at pH 5.5 as compared to a corresponding wild-type Fc region. In some embodiments, the FcRn antagonist comprises or consists of a variant Fc region, or FcRn binding fragment thereof, which binds to FcRn with a higher affinity at pH 6.0 and/or at pH 7.4 as compared to a corresponding wild-type Fc region. In some embodiments, the FcRn antagonist comprises a variant Fc region, or FcRn binding fragment thereof, which binds to FcRn with a higher affinity at both acidic and neutral pH.

In some embodiments, the variant Fc region is derived from the Fc region of any native immunoglobulin. In some embodiments, the native immunoglobulin is a human immunoglobulin. In some embodiments, the immunoglobulin is IgA, IgD, IgE, or IgG. In some embodiments, the immunoglobulin is IgG. In some embodiments, the immunoglobulin is human IgA, human IgD, human IgE, or human IgG. In some embodiments, the immunoglobulin is human IgG. In some embodiments, the IgG is IgG1, IgG2, IgG3, or IgG4. In some embodiments, the human IgG is human IgG1, human IgG2, human IgG3, or human IgG4. In some embodiments, the variant Fc region varies from the human IgG1 Fc region. In some embodiments, the human IgG1 Fc region comprises a G1m1(a), G1m2(x), G1m3(f), or G1m17(z) allotype.

In an embodiment, the variant Fc region, or FcRn binding fragment thereof consists of two Fc domains.

In an embodiment, the variant Fc region comprises or consists of a first Fc domain and a second Fc domain which form a homodimer or heterodimer. In an embodiment, the first Fc domain and/or the second Fc domain comprise amino acids Y, T, E, K, and F at EU positions 252, 254, 256, 433, and 434, respectively. In an embodiment, the first Fc domain and/or the second Fc domain comprise amino acids Y, T, E, K, F, and Y at EU positions 252, 254, 256, 433, 434, and 436, respectively.

In some embodiments, the FcRn antagonists disclosed herein comprise or consist of at least one Fc domain, wherein the amino acid sequence of the at least one Fc domain comprises or consists of the amino acid sequence of SEQ ID NO: 13, provided below in Table 1.

TABLE 1

| | SEQ |
|---|---|
| Amino Acid Sequence | ID NO: |
| X₁X₂X₃X₄X₅X₆PPCPAPELLGGPSVFLFPPKPKDTLYITREPEVTCVVVDVSHE | 13 |
| DPEVKFNWYVDGVEVHNAKTKPREEQYNSTYRVVSVLTVLHQDWLNGKE | |
| YKCKVSNKALPAPIEKTISKAKGQPREPQVYTLPPSRDELTKNQVSLTCLVK | |
| GFYPSDIAVEWESNGQPENNYKTTPPVLDSDGSFFLYSKLTVDKSRWQQGN | |
| VFSCSVMHEALKFHYTQKSLSLSPX₇X₈ | |
| wherein: X₁ is D or absent; X₂ is K or absent; X₃ is T or absent; | |
| X₄ is H or absent; X₅ is T or absent; X₆ is C or absent; X₇ is G | |
| or absent; X₈ is K or absent. | |

In some embodiments, the FcRn antagonists disclosed herein comprise or consist of a variant Fc region comprising or consisting of a dimer of a first Fc domain and a second Fc domain, wherein the amino acid sequence of the first and second Fc domain comprises or consists of the amino acid sequence of SEQ ID NO: 13.

In some embodiments, the FcRn antagonists disclosed herein comprise or consist of an amino acid sequence at least 70%, 75%, 80%, 85%, 90%, 95%, 96%, 97%, 98%, 99%, or 10000 identical to the amino acid sequence of a variant Fc region comprising or consisting of a dimer of a first Fc domain and a second Fc domain, wherein the amino acid sequence of the first and second Fc domain comprises or consists of an amino acid sequence independently selected from the group consisting of SEQ ID NOs: 1-3 and 14-31. In some embodiments, the dimer is a heterodimer or a homodimer.

TABLE 2

| | SEQ |
|---|---|
| Amino Acid Sequence | ID NO: |
| DKTHTCPPCPAPELLGGPSVFLFPPKPKDTLYITREPEVTCVVVDVSHEDPE | 2 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPGK | |
| DKTHTCPPCPAPELLGGPSVFLFPPKPKDTLYITREPEVTCVVVDVSHEDPE | 3 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPG | |
| DKTHTCPPCPAPELLGGPSVFLFPPKPKDTLYITREPEVTCVVVDVSHEDPE | 14 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSP | |
| KTHTCPPCPAPELLGGPSVFLFPPKPKDTLYITREPEVTCVVVDVSHEDPEV | 15 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPGK | |
| KTHTCPPCPAPELLGGPSVFLFPPKPKDTLYITREPEVTCVVVDVSHEDPEV | 16 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPG | |
| KTHTCPPCPAPELLGGPSVFLFPPKPKDTLYITREPEVTCVVVDVSHEDPEV | 17 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSP | |
| THTCPPCPAPELLGGPSVFLFPPKPKDTLYITREPEVTCVVVDVSHEDPEVKF | 18 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPGK | |
| THTCPPCPAPELLGGPSVFLFPPKPKDTLYITREPEVTCVVVDVSHEDPEVKF | 19 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPG | |
| THTCPPCPAPELLGGPSVFLFPPKPKDTLYITREPEVTCVVVDVSHEDPEVKF | 20 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSP | |
| TCPPCPAPELLGGPSVFLFPPKPKDTLYITREPEVTCVVVDVSHEDPEVKFN | 21 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| TCPPCPAPELLGGPSVFLFPPKPKDTLYITREPEVTCVVVDVSHEDPEVKFN | 22 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| TCPPCPAPELLGGPSVFLFPPKPKDTLYITREPEVTCVVVDVSHEDPEVKFN | 23 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| PPCPAPELLGGPSVFLFPPKPKDTLYITREPEVTCVVVDVSHEDPEVKFNWY | 24 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPGK | |
| PPCPAPELLGGPSVFLFPPKPKDTLYITREPEVTCVVVDVSHEDPEVKFNWY | 25 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPG | |
| PPCPAPELLGGPSVFLFPPKPKDTLYITREPEVTCVVVDVSHEDPEVKFNWY | 26 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSP | |
| HTCPPCPAPELLGGPSVFLFPPKPKDTLYITREPEVTCVVVDVSHEDPEVKFN | 27 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| HTCPPCPAPELLGGPSVFLFPPKPKDTLYITREPEVTCVVVDVSHEDPEVKFN | 28 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| HTCPPCPAPELLGGPSVFLFPPKPKDTLYITREPEVTCVVVDVSHEDPEVKFN | 29 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| CPPCPAPELLGGPSVFLFPPKPKDTLYITREPEVTCVVVDVSHEDPEVKFNW | 1 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPG | |
| CPPCPAPELLGGPSVFLFPPKPKDTLYITREPEVTCVVVDVSHEDPEVKFNW | 30 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPGK | |
| CPPCPAPELLGGPSVFLFPPKPKDTLYITREPEVTCVVVDVSHEDPEVKFNW | 31 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSP | |

In an embodiment, the first Fc domain and/or the second Fc domain comprise an amino acid sequence independently selected from the group consisting of SEQ ID NO: 1, SEQ ID NO: 2, and SEQ ID NO: 3. In an embodiment, the first Fc domain and the second Fc domain comprise an amino acid sequence independently selected from the group consisting of SEQ ID NO: 1, SEQ ID NO: 2, and SEQ ID NO: 3.

In an embodiment, the first Fc domain and/or the second Fc domain comprise an amino acid sequence independently selected from the group consisting of SEQ ID NOs: 1, 2, 3, and 30. In an embodiment, the first Fc domain and the second Fc domain comprise an amino acid sequence independently selected from the group consisting of SEQ ID NOs: 1, 2, 3, and 30.

In some embodiments, the FcRn antagonist comprises a population of FcRn antagonist molecules. In some embodiments, an FcRn antagonist comprising a first Fc domain and a second Fc domain comprising an amino acid sequence independently selected from the group consisting of SEQ ID NOs: 1, 2, and 3 is the predominant FcRn antagonist molecule in the population of FcRn antagonist molecules. In some embodiments, an FcRn antagonist comprising a first Fc domain and a second Fc domain comprising an amino acid sequence independently selected from the group consisting of SEQ ID NOs: 1, 2, 3, and 30 is the predominant FcRn antagonist molecule in the population of FcRn antagonist molecules. In some embodiments, the predominant FcRn antagonist molecule makes up at least 50%, 55%, 60%, 65%, 70%, 75%, 80%, 85%, 90%, 95%, 96%, 97%, 98%, or 99% of the population of FcRn antagonist molecules.

In an embodiment, the amino acid sequence of the Fc domains of the variant Fc region comprises the amino acid sequence of SEQ ID NO: 1. In an embodiment, the amino acid sequence of the Fc domains of the variant Fc region consists of the amino acid sequence of SEQ ID NO: 1. In an embodiment, the amino acid sequence of the Fc domains of the variant Fc region comprises the amino acid sequence of SEQ ID NO: 2. In an embodiment, the amino acid sequence of the Fc domains of the variant Fc region consists of the amino acid sequence of SEQ ID NO: 2. In an embodiment, the amino acid sequence of the Fc domains of the variant Fc region comprises the amino acid sequence of SEQ ID NO: 3. In an embodiment, the amino acid sequence of the Fc domains of the variant Fc region consists of the amino acid sequence of SEQ ID NO: 3. In an embodiment, the amino acid sequence of the Fc domains of the variant Fc region comprises the amino acid sequence of SEQ ID NO: 30. In an embodiment, the amino acid sequence of the Fc domains of the variant Fc region consists of the amino acid sequence of SEQ ID NO: 30.

In an embodiment, the FcRn antagonist consists of a variant Fc region, wherein the variant Fc region comprises two Fc domains, wherein the amino acid sequence of each of the Fc domains is independently selected from SEQ ID NO: 1, SEQ ID NO: 2, or SEQ ID NO: 3. In an embodiment, the FcRn antagonist consists of a variant Fc region, wherein the variant Fc region comprises two Fc domains, wherein the amino acid sequence of each of the Fc domains is independently selected from SEQ ID NO: 1, SEQ ID NO: 2, SEQ ID NO: 3, and SEQ ID NO: 30.

In certain embodiments, the variant Fc region is a heterodimer, where the constituent Fc domains are different from each other. Methods of producing Fc heterodimers are known in the art (see e.g., U.S. Pat. No. 8,216,805, which is incorporated by reference herein in its entirety). In an embodiment, the FcRn antagonist consists of a variant Fc region, wherein the variant Fc region consists of two Fc domains which form a heterodimer, wherein the amino acid sequence of each of the Fc domains is independently selected from SEQ ID NO: 1, SEQ ID NO: 2, or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 1 and the amino acid sequence of the second Fc domain consists of or comprises the amino acid sequence of SEQ ID NO: 2 or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 2 and the amino acid sequence of the second Fc domain consists of or comprises the amino acid sequence of SEQ ID NO: 1 or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 3 and the amino acid sequence of the second Fc domain consists of or comprises the amino acid sequence of SEQ ID NO: 1 or SEQ ID NO: 2.

In an embodiment, the FcRn antagonist consists of a variant Fc region, wherein the variant Fc region consists of two Fc domains which form a heterodimer, wherein the amino acid sequence of each of the Fc domains is independently selected from SEQ ID NO: 1, SEQ ID NO: 2, SEQ ID NO: 3, or SEQ ID NO: 30. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 1, and the amino acid sequence of the second Fc domain consists of or comprises the amino acid sequence of SEQ ID NO: 2, SEQ ID NO: 3, or SEQ ID NO: 30. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 2, and the amino acid sequence of the second Fc domain consists of or comprises the amino acid sequence of SEQ ID NO: 1, SEQ ID NO: 3, or SEQ ID NO: 30. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 3, and the amino acid sequence of the second Fc domain consists of or comprises the amino acid sequence of SEQ ID NO: 1, SEQ ID NO: 2, or SEQ ID NO: 30. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 30, and the amino acid sequence of the second Fc domain consists of or comprises the amino acid sequence of SEQ ID NO: 1, SEQ ID NO: 2, or SEQ ID NO: 3.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 1.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 2.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 3.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 30.

In some embodiments, the FcRn antagonist comprises glycanation on one or both of the Fc domains. In some embodiments, the FcRn antagonist molecules comprise glycanation at EU position 297 on one or both of the Fc domains. In some embodiments, the glycanation comprises an N-glycan. In some embodiments, the N-glycan comprises a G0F N-glycan, G1F N-glycan, G2F N-glycan, or G0 N-glycan.

In some embodiments, FcRn antagonist comprises or consists of a population of FcRn antagonists, wherein at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, or at least 57% of the population of Fc domains of the FcRn antagonists comprise galactose. In some embodiments, the population comprises or consists of FcRn antagonists, wherein at least 88%, at least 89%, at least 90%, at least 91%, at least 92%, at least 93%, at least 94%, at least 95%, at least 96%, at least 97%, at least 98%, or at least 99% of the population of Fc domains of the FcRn antagonists comprise fucose.

In some embodiments, the FcRn antagonist lacks an amino acid at EU position 441 of one or both Fc domains. In some embodiments, the FcRn antagonist comprises glycine and lysine at EU positions 440 and 441, respectively. In some embodiments, the FcRn antagonist lacks amino acids at EU positions 440 and 441. In some embodiments, the FcRn antagonist comprises amidated proline at EU position 439. In some embodiments, the FcRn antagonist lacks amino acids at EU positions 440 and 441 and comprises amidated proline at EU position 439.

In some embodiments, the FcRn antagonist comprises aspartate, lysine, threonine, histidine, threonine, and cysteine at EU positions 221, 222, 223, 224, 225, and 226, respectively. In some embodiments, the FcRn antagonist lacks an amino acid at EU positions 221, and comprises lysine, threonine, histidine, threonine, and cysteine at EU positions 222, 223, 224, 225, and 226, respectively. In some embodiments, the FcRn antagonist lacks amino acids at EU positions 221 and 222, and comprises threonine, histidine, threonine, and cysteine at EU positions 223, 224, 225, and 226, respectively. In some embodiments, the FcRn antagonist lacks amino acids at EU positions 221-224, and comprises threonine and cysteine at EU positions 225 and 226, respectively. In some embodiments, the FcRn antagonist lacks amino acids at EU positions 221, 222, 223, 224, 225, and 226.

In some embodiments, the FcRn antagonist is a population of FcRn antagonist molecules. In some embodiments, the population of FcRn antagonist molecules comprises or consists of multiple subpopulations of FcRn antagonist molecules. In some embodiments, the population of FcRn antagonist molecules comprises or consists of 2, 3, 4, 5, 6, 7, 8, 9, 10, 11, 12, 13, 14, 15, 16, 17, 18, 19, or 20 subpopulations. In some embodiments, the population of FcRn antagonist molecules comprises or consists of 2, 3, 4, 5, 6, 7, 8, 9, 10, or 11 subpopulations.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 22, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 19, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 19, respectively, and wherein one asparagine residue in each FcRn antagonist molecule in the fifth subpopulation are deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 16, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one methionine residue or one tryptophan residue in each FcRn antagonist molecule in the eleventh subpopulation is oxidized.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 22, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 19, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 19, respectively, and wherein one asparagine residue in each FcRn antagonist molecule in the fifth subpopulation is deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 16, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein one methionine residue or one tryptophan residue in each FcRn antagonist molecule in the eleventh subpopulation is oxidized.

In some embodiments, the population of FcRn antagonist molecules comprises or consists of the first subpopulation combined with one of the second, third, fourth, fifth, sixth, seventh, eighth, ninth, tenth, or eleventh subpopulations. In some embodiments, the population of FcRn antagonist molecules comprises or consists of the first subpopulation combined with two of the second, third, fourth, fifth, sixth, seventh, eighth, ninth, tenth, or eleventh subpopulations. In some embodiments, the population of FcRn antagonist molecules comprises or consists of the first subpopulation combined with three of the second, third, fourth, fifth, sixth, seventh, eighth, ninth, tenth, or eleventh subpopulations. In some embodiments, the population of FcRn antagonist molecules comprises or consists of the first subpopulation combined with four of the second, third, fourth, fifth, sixth, seventh, eighth, ninth, tenth, or eleventh subpopulations. In some embodiments, the population of FcRn antagonist molecules comprises or consists of the first subpopulation combined with five of the second, third, fourth, fifth, sixth, seventh, eighth, ninth, tenth, or eleventh subpopulations. In some embodiments, the population of FcRn antagonist molecules comprises or consists of the first subpopulation combined with six of the second, third, fourth, fifth, sixth, seventh, eighth, ninth, tenth, or eleventh subpopulations. In some embodiments, the population of FcRn antagonist molecules comprises or consists of the first subpopulation combined with seven of the second, third, fourth, fifth, sixth, seventh, eighth, ninth, tenth, or eleventh subpopulations. In some embodiments, the population of FcRn antagonist molecules comprises or consists of the first subpopulation combined with eight of the second, third, fourth, fifth, sixth, seventh, eighth, ninth, tenth, or eleventh subpopulations. In some embodiments, the population of FcRn antagonist molecules comprises or consists of the first subpopulation combined with nine of the second, third, fourth, fifth, sixth, seventh, eighth, ninth, tenth, or eleventh subpopulations. In some embodiments, the population of FcRn antagonist molecules comprises or consists of the first subpopulation combined with all of the second, third, fourth, fifth, sixth, seventh, eighth, ninth, tenth, or eleventh subpopulations.

In some embodiments, the population comprises or consists of the first and second subpopulations. In some embodiments, the population comprises or consists of the first and third subpopulations. In some embodiments, the population comprises or consists of the first and fourth subpopulations. In some embodiments, the population comprises or consists of the first and fifth subpopulations. In some embodiments, the population comprises or consists of the first and sixth subpopulations. In some embodiments, the population comprises or consists of the first and seventh subpopulations. In some embodiments, the population comprises or consists of the first and eighth subpopulations. In some embodiments, the population comprises or consists of the first and ninth subpopulations. In some embodiments, the population comprises or consists of the first and tenth subpopulations. In some embodiments, the population comprises or consists of the first and eleventh subpopulations. In some embodiments, the populations listed above further comprise or consist of 1, 2, 3, 4, 5, 6, 7, 8, or 9 additional subpopulations. In some embodiments, these additional subpopulations are one or more of those described above.

In some embodiments, the population comprises or consists of the first and seventh, ninth, or eleventh subpopulations. In some embodiments, the population comprises or consists of the first, seventh, ninth, and eleventh subpopulations.

In some embodiments, the first subpopulation makes up at least 40%, at least 45%, at least 50%, at least 55%, at least 60%, at least 65%, at least 70%, at least 75%, at least 80%, at least 85%, or at least 90% of the population of FcRn antagonist molecules. In some embodiments, the first subpopulation makes up about 40%, about 45%, about 50%, about 55%, about 60%, about 65%, about 70%, about 75%, about 80%, about 85%, or about 90% of the population of FcRn antagonist molecules. In some embodiments, the first subpopulation makes up 40%, 45%, 50%, 55%, 60%, 65%, 70%, 75%, 80%, 85%, or 90% of the population of FcRn antagonist molecules. In some embodiments, the first subpopulation makes up 40%-90%, 50%-80%, or 55%-70% of the population of FcRn antagonist molecules. In some embodiments, the first subpopulation makes up 56.9%-68.3% or 59.5%-67.9% of the population of FcRn antagonist molecules.

In some embodiments, the second subpopulation makes up less than 3.0%, less than 2.5%, less than 2.0%, less than 1.5%, less than 1%, or less than 0.5% of the population of FcRn antagonist molecules. In some embodiments, the second subpopulation makes up about 3.0%, about 2.5%, about 2.0%, about 1.5%, about 1%, or about 0.5% of the population of FcRn antagonist molecules. In some embodiments, the second subpopulation makes up 3.0%, 2.5%, 2.0%, 1.5%, 1%, or 0.5% of the population of FcRn antagonist molecules. In some embodiments, the second subpopulation makes up 0.5%-3.0%, 1.0%-2.5%, or 1.0%-2.0% of the population of FcRn antagonist molecules. In some embodiments, the second subpopulation makes up 0.8%-2.0% or 0.8%-2.1% of the population of FcRn antagonist molecules.

In some embodiments, the third subpopulation makes up less than 3.0%, less than 2.5%, less than 2.0%, less than 1.5%, less than 1%, or less than 0.5% of the population of FcRn antagonist molecules. In some embodiments, the third subpopulation makes up about 3.0%, about 2.5%, about 2.0%, about 1.5%, about 1%, or about 0.5% of the population of FcRn antagonist molecules. In some embodiments, the third subpopulation makes up 3.0%, 2.5%, 2.0%, 1.5%, 1%, or 0.5% of the population of FcRn antagonist molecules. In some embodiments, the third subpopulation makes up 0.5%-3.0%, 1.0%-2.5%, or 1.0%-2.0% of the population of FcRn antagonist molecules. In some embodiments, the third subpopulation makes up 1.1%-2.1% or 1.0%-1.9% of the population of FcRn antagonist molecules.

In some embodiments, the fourth subpopulation makes up less than 5%, less than 4%, less than 3%, less than 2%, or less than 1% of the population of FcRn antagonist molecules. In some embodiments, the fourth subpopulation makes up about 5%, about 4%, about 3%, about 2%, or about 1% of the population of FcRn antagonist molecules. In some embodiments, the fourth subpopulation makes up 5%, 4%, 3%, 2%, or 1% of the population of FcRn antagonist molecules. In some embodiments, the fourth subpopulation makes up 1%-5%, 2%-4%, or 2%-3% of the population of FcRn antagonist molecules. In some embodiments, the fourth subpopulation makes up 2.1%-3.2% or 2.0%-3.1% of the population of FcRn antagonist molecules.

In some embodiments, the fifth subpopulation makes up less than 12%, less than 11%, less than 10%, less than 9%, less than 8%, less than 7%, less than 6%, or less than 5% of the population of FcRn antagonist molecules. In some embodiments, the fifth subpopulation makes up about 12%, about 11%, about 10%, about 9%, about 8%, about 7%, about 6%, or about 5% of the population of FcRn antagonist molecules. In some embodiments, the fifth subpopulation makes up 12%, 11%, 10%, 9%, 8%, 7%, 6%, or 5% of the population of FcRn antagonist molecules. In some embodiments, the fifth subpopulation makes up 5%-12%, 6%-10%, or 7%-8% of the population of FcRn antagonist molecules. In some embodiments, the fifth subpopulation makes up 6.8%-9.4% or 6.9%-8.7% of the population of FcRn antagonist molecules.

In some embodiments, the sixth subpopulation makes up less than 17%, less than 16%, less than 15%, less than 14%, less than 13%, less than 12%, less than 11%, less than 10%, less than 9%, less than 8%, less than 7%, or less than 6% of the population of FcRn antagonist molecules. In some embodiments, the sixth subpopulation makes up about 17%, about 16%, about 15%, about 14%, about 13%, about 12%, about 11%, about 10%, about 9%, about 8%, about 7%, or about 6% of the population of FcRn antagonist molecules. In some embodiments, the sixth subpopulation makes up 17%, 16%, 15%, 14%, 13%, 12%, 11%, 10%, 9%, 8%, 7%, or 6% of the population of FcRn antagonist molecules. In some embodiments, the sixth subpopulation makes up 7%-17%, 10%-15%, or 11%-12% of the population of FcRn antagonist molecules. In some embodiments, the sixth subpopulation makes up 7.0%-14.0% or 10.0%-14.4% of the population of FcRn antagonist molecules.

In some embodiments, the seventh subpopulation makes up less than 6.0%, less than 5.5%, less than 5.0%, less than 4.5%, less than 4.0%, less than 3.5%, less than 3.0%, less than 2.5%, less than 2.0%, less than 1.5%, less than 1%, or less than 0.5% of the population of FcRn antagonist molecules. In some embodiments, the seventh subpopulation makes up about 6.0%, about 5.5%, about 5.0%, about 4.5%, about 4.0%, about 3.5%, about 3.0%, about 2.5%, about 2.0%, about 1.5%, about 1%, or about 0.5% of the population of FcRn antagonist molecules. In some embodiments, the seventh subpopulation makes up 6.0%, 5.5%, 5.0%, 4.5%, 4.0%, 3.5%, 3.0%, 2.5%, 2.0%, 1.5%, 1%, or 0.5% of the population of FcRn antagonist molecules. In some embodiments, the seventh subpopulation makes up 0.5%-5.5%, 1.0%-3.0%, or 1.5%-2.5% of the population of FcRn antagonist molecules. In some embodiments, the seventh subpopulation makes up 1.5%-5.5% or 1.4%-4.9% of the population of FcRn antagonist molecules.

In some embodiments, the eighth subpopulation makes up less than 7.5%, less than 7.0%, less than 6.5%, less than 6.0%, less than 5.5%, less than 5.0%, less than 4.5%, less than 4.0%, less than 3.5%, less than 3.0%, or less than 2.5% of the population of FcRn antagonist molecules. In some embodiments, the eighth subpopulation makes up about 7.5%, about 7.0%, about 6.5%, about 6.0%, about 5.5%, about 5.0%, about 4.5%, about 4.0%, about 3.5%, about 3.0%, or about 2.5% of the population of FcRn antagonist molecules. In some embodiments, the eighth subpopulation makes up 7.5%, 7.0%, 6.5%, 6.0%, 5.5%, 5.0%, 4.5%, 4.0%, 3.5%, 3.0%, or 2.5% of the population of FcRn antagonist molecules. In some embodiments, the eighth subpopulation makes up 2.5%-7.5%, 3.0%-5.0%, or 3.5%-4.5% of the population of FcRn antagonist molecules. In some embodiments, the eighth subpopulation makes up 2.9%-7.4% or 3.0%-6.3% of the population of FcRn antagonist molecules.

In some embodiments, the ninth subpopulation makes up less than 3.5%, less than 3.0%, less than 2.5%, less than 2.0%, less than 1.5%, less than 1%, or less than 0.5% of the population of FcRn antagonist molecules. In some embodiments, the ninth subpopulation makes up about 3.5%, about 3.0%, about 2.5%, about 2.0%, about 1.5%, about 1%, or about 0.5% of the population of FcRn antagonist molecules. In some embodiments, the ninth subpopulation makes up 3.5%, 3.0%, 2.5%, 2.0%, 1.5%, 1%, or 0.5% of the population of FcRn antagonist molecules. In some embodiments, the ninth subpopulation makes up 0.5%-3.5%, 1.5%-2.0%, or 1.0%-1.5% of the population of FcRn antagonist molecules. In some embodiments, the ninth subpopulation makes up 0.4%-3.2% or 0.5%-2.6% of the population of FcRn antagonist molecules.

In some embodiments, the tenth subpopulation makes up less than 2.0%, less than 1.5%, less than 1%, or less than 0.5% of the population of FcRn antagonist molecules. In some embodiments, the tenth subpopulation makes up about 2.0%, about 1.5%, about 1%, or about 0.5% of the population of FcRn antagonist molecules. In some embodiments, the tenth subpopulation makes up 2.0%, 1.5%, 1%, or 0.5% of the population of FcRn antagonist molecules. In some embodiments, the tenth subpopulation makes up 0.5%-2.0%, 0.5%-1.5%, or 1.0%-1.5% of the population of FcRn antagonist molecules.

In some embodiments, the eleventh subpopulation makes up less than 2.0%, less than 1.5%, less than 1%, or less than 0.5% of the population of FcRn antagonist molecules. In some embodiments, the eleventh subpopulation makes up about 2.0%, about 1.5%, about 1%, or about 0.5% of the population of FcRn antagonist molecules. In some embodiments, the eleventh subpopulation makes up 2.0%, 1.5%, 1%, or 0.5% of the population of FcRn antagonist molecules. In some embodiments, the eleventh subpopulation makes up 0.5%-2.0%, 0.5%-1.5%, or 1.0%-1.5% of the population of FcRn antagonist molecules.

In some embodiments, the population of FcRn antagonist molecules comprises one or more of the FcRn antagonists described herein. In some embodiments, the FcRn antagonist is any of those described in U.S. Patent Application No. 63/383,599, filed on Nov. 14, 2022, incorporated herein by reference in its entirety. In some embodiments, the FcRn antagonist is a population of FcRn antagonists as described in U.S. Patent Application No. 63/383,599, filed on Nov. 14, 2022, incorporated herein by reference in its entirety.

In an embodiment, the FcRn antagonist is efgartigimod (CAS Registry No. 1821402-21-4). The term “efgartigimod” as used herein is interchangeable with “efgartigimod alfa.” In some embodiments, efgartigimod is efgartigimod alfa-fcab.

In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161).

In an embodiment, an antibody that binds specifically to FcRn and inhibits the binding of the Fc region of immunoglobulin to FcRn is nipocalimab, also known as M281. Nipocalimab is a full-length “Fc dead” IgG1 monoclonal antibody. Nipocalimab has been administered as an intravenous infusion in Phase 2 clinical trials for the treatment of myasthenia gravis (MG), warm autoimmune hemolytic anemia (WAIHA), and hemolytic disease of fetus and newborn (HDFN). Nipocalimab comprises the light chain (SEQ ID NO: 4) and heavy chain (SEQ ID NO: 5) sequences set forth in Table 3 below (VL region of SEQ NO: 4 and VH region of SEQ ID NO: 5 are underlined):

TABLE 3
Heavy chain and light chain sequences of nipocalimab

| SEQ ID NO: | Amino Acid Sequence |
|---|---|
| 4 | QSALTQPASVSGSPGQSITISCTGTGSDVGSYNLVSWYQQHPGKAP |
| (light chain | KLMIYGDSERPSGVSNRFSGSKSGNTASLTISGLQAEDEADYYCSSY |
| sequence; | AGSGIYVFGTGTKVTVLGQPKAAPSVTLFPPSSEELQANKATLVCLI |
| VL region | SDFYPGAVTVAWKADSSPVKAGVETTTPSKQSNNKYAASSYLSLT |
| underlined) | PEQWKSHKSYSCQVTHEGSTVEKTVAPTECS |
| 5 | EVQLLESGGGLVQPGGSLRLSCAASGFTFSTYAMGWVRQAPGKGL |
| (heavy chain | EWVSSIGASGSQTRYADSVKGRFTISRDNSKNTLYLQMNSLRAEDT |
| sequence; | AVYYCARLAIGDSYWGQGTMVTVSSASTKGPSVFPLAPSSKSTSGG |
| VH region | TAALGCLVKDYFPEPVTVSWNSGALTSGVHTFPAVLQSSGLYSLSS |
| underlined) | VVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFN |
| | WYVDGVEVHNAKTKPREEQYASTYRVVSVLTVLHQDWLNGKEY |
| | KCKVSNKALPAPIEKTISKAKGQPREPQVYTLPPSREEMTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPG |

In an embodiment, an antibody that binds specifically to FcRn and inhibits the binding of the Fc region of immunoglobulin to FcRn is rozanolixizumab, also known as UCB 7665. Rozanolixizumab is a full-length humanized IgG4 monoclonal antibody. Rozanolixizumab has been administered as a subcutaneous infusion in ongoing clinical trials for MG, immune thrombocytopenia (FTP), and chronic inflammatory demyelinating polyneuropathy (CIDP). Rozanolixizumab comprises the light chain (SEQ ID NO: 6) and heavy chain (SEQ ID NO: 7) sequences set forth in Table 4 below (VL region of SEQ NO: 6 and VH region of SEQ ID NO: 7 are underlined):

TABLE 4
Heavy chain and light chain sequences of rozanolixizumab

| SEQ ID NO: | Amino Acid Sequence |
|---|---|
| 6 | DIQMTQSPSSLSASVGDRVTITCKSSQSLVGASGKTYLYWLFQKPG |
| (light chain | KAPKRLIYLVSTLDSGIPSRFSGSGSGTEFTLTISSLQPEDFATYYCLQ |
| sequence; | GTHFPHTFGQGTKLEIKRTVAAPSVFIFPPSDEQLKSGTASVVCLLN |
| VL region | NFYPREAKVQWKVDNALQSGNSQESVTEQDSKDSTYSLSSTLTLS |
| underlined) | KADYEKHKVYACEVTHQGLSSPVTKSFNRGEC |
| 7 | EVPLVESGGGLVQPGGSLRLSCAVSGFTFSNYGMVWVRQAPGKGL |
| (heavy chain | EWVAYIDSDGDNTYYRDSVKGRFTISRDNAKSSLYLQMNSLRAED |
| sequence; | TAVYYCTTGIVRPFLYWGQGTLVTVSSASTKGPSVFPLAPCSRSTSE |
| VH region | STAALGCLVKDYFPEPVTVSWNSGALTSGVHTFPAVLQSSGLYSLS |
| underlined) | SVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCK |
| | VSNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMHEALHNHYTQKSLSLSLGK |

In an embodiment, an antibody that binds specifically to FcRn and inhibits the binding of the Fc region of immunoglobulin to FcRn is orilanolimab, also known as SYNT001. Orilanolimab is another full-length humanized IgG4 monoclonal antibody. Orilanolimab has been administered as an intravenous infusion in Phase 2 clinical trials for treatment of WAIHA. Orilanolimab comprises the light chain (SEQ ID NO: 8) and heavy chain (SEQ ID NO: 9) sequences set forth in Table 5 below (VL region of SEQ NO: 8 and VH region of SEQ ID NO: 9 are underlined):

TABLE 5
Heavy chain and light chain sequences of orilanolimab

| SEQ ID NO: | Amino Acid Sequence |
|---|---|
| 8 | DIQMTQSPSSLSASVGDRVTITCKASDHINNWLAWYQQKPGQAPR |
| (light chain | LLISGATSLETGVPSRFSGSGTGKDYTLTISSLQPEDFATYYCQQYW |
| sequence; | STPYTFGGGTKVEIKRTVAAPSVFIFPPSDEQLKSGTASVVCLLNNF |
| VL region | YPREAKVQWKVDNALQSGNSQESVTEQDSKDSTYSLSSTLTLSKA |
| underlined) | DYEKHKVYACEVTHQGLSSPVTKSFNRGEC |
| 9 | QVQLVQSGAELKKPGASVKLSCKASGYTFTSYGISWVKQATGQGL |
| (heavy chain | EWIGEIYPRSGNTYYNEKFKGRATLTADKSTSTAYMELRSLRSEDS |
| sequence; | AVYFCARSTTVRPPGIWGTGTTVTVSSASTKGPSVFPLAPCSRSTSE |
| VH region | STAALGCLVKDYFPEPVTVSWNSGALTSGVHTFPAVLQSSGLYSLS |
| underlined) | SVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCK |
| | VSNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMHEALHNHYTQKSLSLSLG |

In an embodiment, an antibody that binds specifically to FcRn and inhibits the binding of the Fc region of immunoglobulin to FcRn is batoclimab, also known as IMVT1401/RVT1401/HBM9161. Batoclimab is another full-length “Fc dead” IgG1 monoclonal antibody. Batoclimab has been administered as a subcutaneous injection in ongoing Phase 2 clinical trials for treatment of MG and Graves' ophthalmopathy. Batoclimab comprises the light chain (SEQ ID NO: 10) and heavy chain (SEQ ID NO: 11) sequences set forth in Table 6 below (VL region of SEQ NO: 10 and VH region of SEQ ID NO: 11 are underlined):

TABLE 6
Heavy chain and light chain sequences of batoclimab

| SEQ ID NO: | Amino Acid Sequence |
|---|---|
| 10 | SYVLTQSPSVSVAPGQTARITCGGNNIGSKSVHWYQQKPGQAPVL |
| (light chain | VVYDDSDRPSGIPERFSASNSGNTATLTISRVEAGDEADYYCQVWD |
| sequence; | SSSDHVVFGGGTKLTVLGQPKAAPSVTLFPPSSEELQANKATLVCLI |
| VL region | SDFYPGAVTVAWKADSSPVKAGVETTTPSKQSNNKYAASSYLSLT |
| underlined) | PEQWKSHRSYSCQVTHEGSTVEKTVAPTECS |
| 11 | QLLLQESGPGLVKPSETLSLTCTVSGGSLSSSFSYWVWIRQPPGKGL |
| (heavy chain | EWIGTIYYSGNTYYNPSLKSRLTISVDTSKNHFSLKLSSVTAADTAV |
| sequence; | YYCARRAGILTGYLDSWGQGTLVTVSSASTKGPSVFPLAPSSKSTS |
| VH region | GGTAALGCLVKDYFPEPVTVSWNSGALTSGVHTFPAVLQSSGLYS |
| underlined) | LSSVVTVPSSSLGTQTYICNVNHKPSNTKVDKRVEPKSCDKTHTCP |
| | PCPAPEAAGGPSVFLFPPKPKDTLMISRTPEVTCVVVDVSHEDPEVK |
| | FNWYVDGVEVHNAKTKPREEQYNSTYRVVSVLTVLHQDWLNGK |
| | EYKCKVSNKALPAPIEKTISKAKGQPREPQVYTLPPSREEMTKNQV |
| | SLTCLVKGFYPSDIAVEWESNGQPENNYKTTPPVLDSDGSFFLYSK |
| | LTVDKSRWQQGNVFSCSVMHEALHNHYTQKSLSLSPG |

### Pharmaceutical Compositions

In an aspect, the instant disclosure provides pharmaceutical compositions comprising an FcRn antagonist for use in methods of treating LN. In certain embodiments, these compositions comprise or consist of a variant Fc region, or FcRn-binding fragment thereof, that binds specifically to FcRn, particularly human FcRn, with increased affinity and reduced pH dependence relative to a native Fc region. In other embodiments, the FcRn antagonist composition is an antibody or antigen-binding fragment thereof that binds specifically to FcRn via its antigen binding domain and inhibits the binding of Fc region of immunoglobulin to FcRn. In general, these FcRn antagonists inhibit the binding of Fc-containing agents (e.g., antibodies and immunoadhesins) to FcRn in vivo, which results in an increased rate of degradation of the Fc-containing agents and, concomitantly, a reduced serum level of these agents.

In an embodiment, the FcRn antagonist is efgartigimod. Efgartigimod (ARGX-113) is a modified human immunoglobulin (Ig) gamma (IgG) 1-derived Fc of the za allotype that binds with nanomolar affinity to human FcRn. Efgartigimod encompasses the IgG1 Fc-region and has been engineered using ABDEG™ technology to increase its affinity for FcRn at both physiological and acidic pH. The increased affinity for FcRn of efgartigimod at both acidic and physiological pH results in a blockage of FcRn-mediated recycling of IgGs.

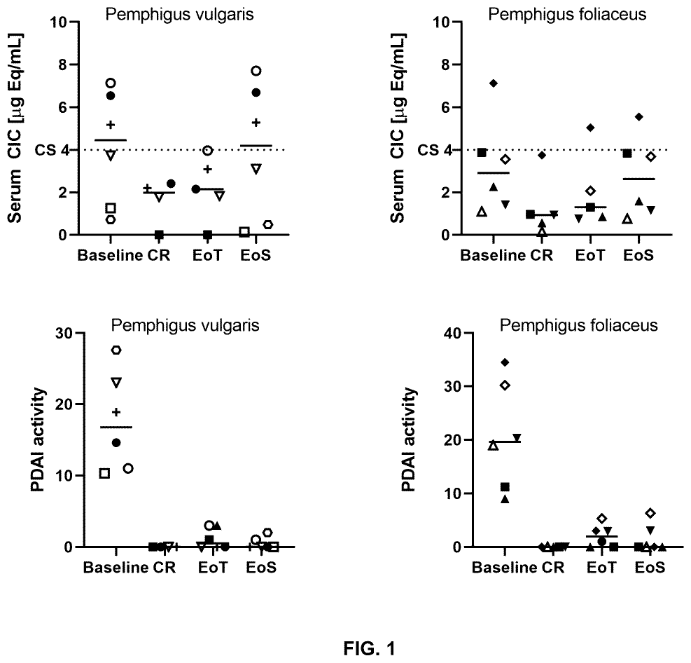

Due to its increased affinity for FcRn at both acidic and neutral pH, efgartigimod blocks the FcRn/IgG complex from forming, which results in degradation of endogenous IgGs, including autoantibodies that cause IgG-mediated autoimmune diseases. This blocking of FcRn by efgartigimod results in a rapid and profound reduction in autoantibody levels, which underlies the therapeutic strategy for the treatment of autoimmune indications where IgG autoantibodies are expected to have a central role in the disease pathology. Without wishing to be bound by theory, efgartigimod may act to prevent the onset or progression of LN by reducing circulating immune complexes, preventing their deposition in glomeruli. Indeed, efgartigimod has been shown to reduce circulating immune complexes (Example 1; FIG. 1).

Efgartigimod is under development for both the intravenous (IV) and subcutaneous (SC) administration route.

For IV administration, in certain embodiments, efgartigimod may be administered in a formulation comprising sodium phosphate, sodium chloride, L-arginine hydrochloride, and polysorbate 80. In certain embodiments, efgartigimod may be administered in a formulation comprising about 25 mM sodium phosphate, about 100 mM sodium chloride, and about 150 mM L-arginine hydrochloride (pH 6.7), with about 0.02% (w/v) polysorbate 80. In certain embodiments, efgartigimod may be administered in a formulation comprising 25 mM sodium phosphate, 100 mM sodium chloride, and 150 mM L-arginine hydrochloride (pH 6.7), with 0.02% (w/v) polysorbate 80. In certain embodiments, efgartigimod may be administered in a formulation comprising about 25 mM sodium phosphate, about 100 mM sodium chloride, and about 150 mM L-arginine hydrochloride (pH 6.7), with about 0.02% (w/v) polysorbate 80, via intravenous infusion in a total volume of about 250 mL over a period of about 2 hours. In certain embodiments, efgartigimod may be administered in a formulation comprising 25 mM sodium phosphate, 100 mM sodium chloride, and 150 mM L-arginine hydrochloride (pH 6.7), with 0.02% (w/v) polysorbate 80, via intravenous infusion in a total volume of 250 mL over a period of 2 hours. See e.g., WO2019110823A1, which is incorporated by reference herein in its entirety.

In certain embodiments, efgartigimod may be administered in a formulation comprising an aqueous solution comprising about 25 mM sodium phosphate, about 100 mM sodium chloride, and about 150 mM L-arginine hydrochloride with a pH of about 6.7, with about 0.02% (w/v) polysorbate 80, diluted for intravenous infusion to a total volume of about 125 mL over a period of about 1 hour. In certain embodiments, efgartigimod may be administered in a formulation comprising an aqueous solution comprising 25 mM sodium phosphate, 100 mM sodium chloride, and 150 mM L-arginine hydrochloride with a pH of 6.7, with 0.02% (w/v) polysorbate 80, diluted for intravenous infusion to a total volume of 125 mL over a period of 1 hour.

In certain embodiments, efgartigimod may be administered in a formulation comprising an aqueous solution comprising about 4 mM sodium phosphate, about 146 mM sodium chloride, about 24 mM L-arginine, and about 0.0032% (w/v) polysorbate 80, with a pH of about 6.7. This formulation is administered via intravenous infusion in a total volume of about 125 mL over a period of about 1 hour. In certain embodiments, efgartigimod may be administered in a formulation comprising an aqueous solution comprising 4 mM sodium phosphate, 146 mM sodium chloride, 24 mM L-arginine, and 0.0032% (w/v) polysorbate 80, with a pH of 6.7. This formulation is administered via intravenous infusion in a total volume of 125 mL over a period of 1 hour.

In certain embodiments, efgartigimod is administered via IV infusion and is provided in a sterile, colorless, clear concentrate solution at a concentration of about 20 mg/mL. In certain embodiments, efgartigimod is administered via IV infusion and is provided in a sterile, colorless, clear concentrate solution at a concentration of 20 mg/mL.

In certain embodiments, efgartigimod is administered via IV infusion and is provided in a vial (e.g., a single-dose vial). In certain embodiments, a vial of efgartigimod contains about 400 mg of efgartigimod at a concentration of about 20 mg/mL. In certain embodiments, a vial of efgartigimod contains 400 mg of efgartigimod at a concentration of 20 mg/mL. In certain embodiments, each mL of solution in a vial of efgartigimod contains about 31.6 mg L-arginine hydrochloride, about 0.2 mg polysorbate 80, about 5.8 mg sodium chloride, about 2.4 mg sodium phosphate dibasic anhydrous, about 1.1 mg sodium phosphate monobasic monohydrate, and water for injection, USP, at a pH of about 6.7. In certain embodiments, each mL of solution in a vial of efgartigimod contains 31.6 mg L-arginine hydrochloride, 0.2 mg polysorbate 80, 5.8 mg sodium chloride, 2.4 mg sodium phosphate dibasic anhydrous, 1.1 mg sodium phosphate monobasic monohydrate, and water for injection, USP, at a pH of 6.7.

In certain embodiments, for patients weighing under 120 kg, efgartigimod is administered at a dose of about 10 mg/kg as an IV infusion. In certain embodiments, for patients weighing under 120 kg, efgartigimod is administered at a dose of about 10 mg/kg as an IV infusion over about one hour. In certain embodiments, for patients weighing under 120 kg, efgartigimod is administered at a dose of about 10 mg/kg as an IV infusion over about one hour once weekly. In certain embodiments, for patients weighing under 120 kg, efgartigimod is administered at a dose of about 10 mg/kg as an IV infusion over about one hour once weekly for about 4 weeks. In certain embodiments, for patients weighing under 120 kg, efgartigimod is administered at a dose of 10 mg/kg as an IV infusion. In certain embodiments, for patients weighing under 120 kg, efgartigimod is administered at a dose of 10 mg/kg as an IV infusion over one hour. In certain embodiments, for patients weighing under 120 kg, efgartigimod is administered at a dose of 10 mg/kg as an IV infusion over one hour once weekly. In certain embodiments, for patients weighing under 120 kg, efgartigimod is administered at a dose of 10 mg/kg as an IV infusion over one hour once weekly for 4 weeks. In certain embodiments, for patients weighing 120 kg or more, efgartigimod is administered at a dose of about 1200 mg per IV infusion. In certain embodiments, for patients weighing 120 kg or more, efgartigimod is administered at a dose of 1200 mg per IV infusion.

For SC administration, in certain embodiments, efgartigimod may be administered alone. Alternatively, for SC administration, in certain embodiments, efgartigimod may be administered co-formulated with hyaluronidase, for example, in particular, rHuPH20. The co-formulated material will allow SC dosing of larger volumes.

In some embodiments, efgartigimod may be administered in a formulation comprising an aqueous solution comprising about 20 mM L-histidine, about 100 mM sodium chloride, about 60 mM sucrose, about 10 mM L-methionine, and about 0.04% (w/v) polysorbate 20, wherein the composition has a pH of about 6.0. In some embodiments, the formulation comprises about 180 mg/ml efgartigimod. In some embodiments, efgartigimod may be administered in a formulation comprising an aqueous solution comprising 20 mM L-histidine, 100 mM sodium chloride, 60 mM sucrose, 10 mM L-methionine, and 0.04% (w/v) polysorbate 20, wherein the composition has a pH of 6.0. In some embodiments, the formulation comprises 180 mg/ml efgartigimod.

In some embodiments, efgartigimod may be administered in a formulation comprising an aqueous solution comprising about 20 mM L-histidine, about 50 mM L-arginine, about 100 mM sodium chloride, about 60 mM sucrose, about 10 mM L-methionine, and about 0.04% (w/v) polysorbate 80, wherein the composition has a pH of about 6.0. In some embodiments, the formulation comprises about 200 mg/ml efgartigimod. In some embodiments, efgartigimod may be administered in a formulation comprising an aqueous solution comprising 20 mM L-histidine, 50 mM L-arginine, 100 mM sodium chloride, 60 mM sucrose, 10 mM L-methionine, and 0.04% (w/v) polysorbate 80, wherein the composition has a pH of 6.0. In some embodiments, the formulation comprises 200 mg/ml efgartigimod. rHuPH20 is the active ingredient of Halozyme's commercial product HYLENEX® recombinant (hyaluronidase human injection), referred to as HYLENEX®, which was approved by FDA for marketed use in the U.S. in December 2005. HYLENEX® is a tissue permeability modifier indicated as an adjuvant in SC fluid administration for achieving hydration, to increase the dispersion and absorption of other injected drugs, and in SC urography, for improving resorption of radiopaque agents.

rHuPH20 is a recombinant enzyme human hyaluronidase produced by genetically engineered Chinese hamster ovary (CHO) cells containing a deoxyribonucleic plasmid encoding a soluble fragment of human hyaluronidase (posterior head protein 20 [PH20]).

The HZ202 rHuPH20 DS is currently registered in HYLENEX® and other biologic drug products co-formulated with rHuPH20 DS. As such, in certain embodiments HZ202 rHuPH20 DS is used in the efgartigimod/rHuPH20 co-formulated product for SC administration (i.e., efgartigimod PH20 SC).

Provided in the co-formulations, combinations, uses and methods herein are soluble hyaluronidases. Soluble hyaluronidases include any that, upon expression, are secreted from a cell and exist in soluble form. Such soluble hyaluronidases include, but are not limited to, bacterial soluble hyaluronidases, non-human soluble hyaluronidases, such as bovine PH20 and ovine PH20, human soluble PH20, and variants thereof. Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g., DG44 CHO cells).

rHuPH20 refers to the composition produced upon expression in a cell, such as a CHO cell, of nucleic acid encoding residues 36-482 of SEQ ID NO:32, generally linked to the native or a heterologous signal sequence (residues 1-35 of SEQ ID NO:32). rHuPH20 is produced by expression of a nucleic acid molecule, such as encoding amino acids 1-482 (set forth in SEQ ID NO:32) in a mammalian cell. Translational processing removes the 35 amino acid signal sequence. As produced in the culture medium there is heterogeneity at the C-terminus such that the product, designated rHuPH20, includes a mixture of species that can include any one or more of the polypeptides 36-480, 36-481, and 36-482 of SEQ ID NO:32, and some shorter polypeptides, in various abundance. Typically, rHuPH20 is produced in cells that facilitate correct N-glycosylation to retain activity, such as CHO cells (e.g., DG44 CHO cells). In some embodiments, one of the most abundant species is the 446 amino acid polypeptide corresponding to residues 36-481 of SEQ ID NO:32. Also included are polypeptides that are soluble or secreted upon expression in a mammalian cell and have at least 90%, 91%, 92%, 93%, 94%, 95%, 96%, 97%, 98%, 99%, or more sequence identity with residues 36-482 of SEQ ID NO:32.

In some embodiments, the pharmaceutical formulation comprises an FcRn antagonist in an amount from about 20 mg to about 20,000 mg. In some embodiments, the pharmaceutical formulation comprises an FcRn antagonist in an amount from about 200 mg to about 20,000 mg. In some embodiments, the pharmaceutical formulation comprises an FcRn antagonist in an amount from about 300 mg to about 6000 mg. In some embodiments, the pharmaceutical formulation comprises an FcRn antagonist in an amount from about 750 mg to about 3000 mg. In some embodiments, the pharmaceutical formulation comprises an FcRn antagonist in an amount from about 1000 mg to about 2500 mg. In some embodiments, the pharmaceutical formulation comprises an FcRn antagonist in an amount from about 1000 mg to about 2000 mg.

In some embodiments, the pharmaceutical formulation comprises an FcRn antagonist in an amount from 20 mg to 20,000 mg. In some embodiments, the pharmaceutical formulation comprises an FcRn antagonist in an amount from 200 mg to 20,000 mg. In some embodiments, the pharmaceutical formulation comprises an FcRn antagonist in an amount from 300 mg to 6000 mg. In some embodiments, the pharmaceutical formulation comprises an FcRn antagonist in an amount from 750 mg to 3000 mg. In some embodiments, the pharmaceutical formulation comprises an FcRn antagonist in an amount from 1000 mg to 2500 mg. In some embodiments, the pharmaceutical formulation comprises an FcRn antagonist in an amount from 1000 mg to 2000 mg.

In some embodiments, the pharmaceutical formulation comprises about 1000 mg or about 2000 mg of an FcRn antagonist. In some embodiments, the pharmaceutical formulation comprises 1000 mg or 2000 mg of an FcRn antagonist. In some embodiments, the FcRn antagonist is efgartigimod.

In some embodiments, the pharmaceutical formulation comprises efgartigimod in an amount from about 800 mg to about 1200 mg. In some embodiments, the pharmaceutical formulation comprises about 1000 mg efgartigimod. In some embodiments, the pharmaceutical formulation comprises 1000 mg efgartigimod.

In some embodiments, the pharmaceutical formulation comprises from about 10 mg/mL to about 200 mg/mL efgartigimod. In some embodiments, the pharmaceutical formulation comprises from 10 mg/mL to 200 mg/mL efgartigimod.

In some embodiments, the pharmaceutical formulation comprises about 20 mg/mL efgartigimod. In some embodiments, the pharmaceutical formulation comprises 20 mg/mL efgartigimod.

In some embodiments, the pharmaceutical formulation comprises about 180 mg/ml efgartigimod. In some embodiments, the pharmaceutical formulation comprises 180 mg/ml efgartigimod.

In some embodiments, the pharmaceutical formulation further comprises hyaluronidase. In some embodiments, the hyaluronidase is recombinant human hyaluronidase PH20 (rHuPH20).

The hyaluronidase can be present in the pharmaceutical formulation in any suitable amount. In an embodiment, the amount of hyaluronidase enzyme is from about 1000 U/ml to about 3000 U/ml. In an embodiment, the amount of hyaluronidase enzyme is about 1000 U/mL, about 1500 U/mL, about 2000 U/mL, about 2500 U/mL, or about 3000 U/mL. In an embodiment, the amount of hyaluronidase enzyme is 2000 U/mL.

In some embodiments, the rHuPH20 is present in the pharmaceutical formulation in an amount of about 11,000 U. In some embodiments, the rHuPH20 is present in the pharmaceutical formulation in an amount of 11,000 U.

In some embodiments, the pharmaceutical formulation comprises at least about 5 U to at least about 100,000 U of an endoglycosidase hydrolase enzyme. In some aspects, the pharmaceutical formulation comprises at least about 5 U, at least about 10 U, at least about 20 U, at least about 30 U, at least about 40 U, at least about 50 U, at least about 75 U, at least about 100 U, at least about 200 U, at least about 300 U, at least about 400 U, at least about 500 U, at least about 750 U, at least about 1000 U, at least about 2000 U, at least about 3000 U, at least about 4000 U, at least about 5000 U, at least about 6000 U, at least about 7000 U, at least about 8000 U, at least about 9000 U, at least about 10,000 U, at least about 20,000 U, at least about 30,000 U, at least about 40,000 U, at least about 50,000 U, at least about 60,000 U, at least about 70,000 U, at least about 80,000 U, at least about 90,000 U, or at least about 100,000 U of an endoglycosidase hydrolase enzyme.

In some embodiments, the pharmaceutical formulation comprises about 20,000 U of an endoglycosidase hydrolase enzyme. In some embodiments, the pharmaceutical formulation comprises at least about 500 U/mL to at least about 5000 U/mL of an endoglycosidase hydrolase enzyme. In some embodiments, the pharmaceutical formulation comprises at least about 1500 U/mL, at least about 1600 U/mL, at least about 1700 U/mL, at least about 1800 U/mL, at least about 1900 U/mL, at least about 2000 U/mL, at least about 2100 U/mL, at least about 2200 U/mL, at least about 2300 U/mL, at least about 2400 μM, at least about 2500 μM, at least about 3000 μM, at least about 3500 μM, at least about 4000 μM, at least about 4500 U/mL, or at least about 5000 U/mL of an endoglycosidase hydrolase enzyme. In some embodiments, the pharmaceutical formulation comprises about 2000 U/mL of an endoglycosidase hydrolase enzyme.

In some embodiments, the endoglycosidase hydrolase enzyme cleaves hyaluronic acid at a hexosaminidic β (1-4) or (1-3) linkage. In some embodiments, the endoglycosidase hydrolase enzyme comprises a catalytic domain of hyaluronidase PH-20 (HuPH20), HYAL1, HYAL2, HYAL3, HYAL4, or HYALPS1. In some embodiments, the endoglycosidase hydrolase enzyme comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to amino acids 36-490 of SEQ ID NO: 32. In some embodiments, the endoglycosidase hydrolase enzyme comprises a hyaluronidase. In some embodiments, the endoglycosidase hydrolase enzyme comprises a hyaluronidase selected from the group consisting of HuPH20, HYAL1, HYAL2, HYAL3, HYAL4, any variant, and any isoform thereof. In some embodiments, the endoglycosidase hydrolase enzyme comprises rHuPH20 or a fragment thereof.

In some embodiments, the endoglycosidase hydrolase enzyme comprises a modified hyaluronidase comprising one or more amino acid substitutions relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase comprising one or more amino acid substitution in an alpha-helix region relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase comprising one or more amino acid substitution in linker region relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase, wherein one or more N-terminal and/or C-terminal amino acids are deleted relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified rHuPH20, wherein the modified rHuPH20 comprises: i. one or more amino acid substitution in an alpha-helix region, a linker region, or both an alpha-helix region and a linker region relative to wild-type rHuPH20; ii. deletion of one or more N-terminal amino acid, one or more C-terminal amino acid, or one or more N-terminal amino acid and one or more C-terminal amino acid relative to wild-type rHuPH20; or iii. both (i) and (ii).

“Hyaluronidase,” as used herein, refers to an enzyme capable of catalyzing the cleavage of hyaluronan. Hyaluronan is a repeating polymer of N-acetyl-glucosamine and glucuronic acid, which is present in the subcutaneous space and contributes to the soluble gel-like component of the extracellular matrix of the skin and is restored by rapid turnover (resynthesis). In some embodiments, the hyaluronidase comprises rHuPH20, which is a glycosylated 447-amino acid single chain polypeptide that depolymerizes hyaluronan in the subcutaneous space locally at the site of injection in the skin. Depolymerization of hyaluronan by hyaluronidase is accomplished by hydrolysis of the polysaccharide polymer. Depolymerization of hyaluronan results in a transient reduction in the viscosity of the gel-like phase of the extracellular matrix and increased hydraulic conductance that facilitates the dispersion and absorption of the coadministered therapeutic agent. Thus, a hyaluronidase, e.g., rHuPH20, can improve the speed and ease of subcutaneous delivery of injectable biologics and drugs by acting as a permeation enhancer. In certain embodiments, the hyaluronidase comprises ENHANZE™.

In any of the above embodiments, the pharmaceutical formulation may be a unit dosage form.

In an embodiment, the unit dosage form comprises the FcRn antagonist as a dry formulation for dissolution such as a lyophilized powder, freeze-dried powder, or water-free concentrate. In an embodiment, the dry formulation is comprised in a hermetically sealed container such as a vial, an ampoule, or a sachet.

In an embodiment, the unit dosage form comprises the FcRn antagonist as a liquid formulation, e.g., injection or infusion solution. In an embodiment, the liquid formulation is comprised in a hermetically sealed container such as a vial, a sachet, a pre-filled syringe, a pre-filled autoinjector, or a cartridge for a reusable syringe or applicator.

In an embodiment, the unit dosage per vial may contain 0.5 ml, 1 ml, 2 ml, 3 ml, 4 ml, 5 ml, 6 ml, 7 ml, 8 ml, 9 ml, 10 ml, 15 ml, or 20 ml of an FcRn antagonist ranging from about 500 to about 2500 mg or from about 1000 mg to about 2000 mg. In an embodiment, these preparations can be adjusted to a desired concentration by adding a sterile diluent to each vial.

The formulations disclosed herein include bulk drug compositions useful in the manufacture of pharmaceutical compositions (e.g., compositions that are suitable for administration to a subject or patient) which can be used in the preparation of unit dosage forms. In an embodiment, a composition of the invention is a pharmaceutical composition. Such compositions comprise a prophylactically or therapeutically effective amount of one or more prophylactic or therapeutic agents (e.g., an FcRn antagonist of the invention or other prophylactic or therapeutic agent), and a pharmaceutically acceptable carrier. In an embodiment, the pharmaceutical compositions are formulated to be suitable for subcutaneous administration to a subject.

### Methods

In an aspect, methods for treating LN using an FcRn antagonist are provided. In certain embodiments, the FcRn antagonist is efgartigimod. An important goal and feature of the methods disclosed herein is prevention of progression to end-stage renal disease (ESRD) in LN patients. Other goals and features of the methods disclosed herein include, but are not limited to, patient survival, long-term preservation of kidney function, prevention of disease flares, prevention of organ damage, management of comorbidities, and improvement in disease-related quality of life. Effective treatment of LN using an FcRn antagonist may include at least one of the elements of the group consisting of: early reduction in UPCR, improvement in Systemic Lupus Erythematosus Disease Activity Index (SLEDAI)-2K score, and/or improvement in estimated glomerular filtration rate (eGFR). In some embodiments, the UPCR is calculated by dividing the level of protein (mg/dl) in a spot urine test by the creatinine level (mg/dl). The SLEDAI-2K is a questionnaire that determines disease activity in lupus. In some embodiments, a subject that is treated according to the methods provided herein experiences a complete renal response (CRR). In some embodiments, a subject that is treated according to the methods provided herein experiences a modified complete renal response (mCRR). In some embodiments, a subject that is treated according to the methods provided herein experiences a partial renal response (PRR). In some embodiments, a subject that is treated according to the methods provided herein experiences a reduction in circulating immune complexes. In some embodiments, the circulating immune complexes are selected from the group consisting of C3, C4, CH50, and C1q-binding circulating immune complexes.

As used herein, a complete renal response (CRR) in a subject is defined as eGFR ≥60 mL/min/1.73 m² or no confirmed decrease from the baseline eGFR value of >20%, and UPCR ≤0.5 mg/mg.

As used herein, a modified complete renal response (mCRR) in a subject is defined as eGFR ≥90 mL/min/1.73 m² or no confirmed decrease from the baseline eGFR value of ≥10%, and UPCR <0.5 mg/mg.

As used herein, a partial renal response (PRR) in a subject is defined as eGFR ≥60 mL/min/1.73 m² or no confirmed decrease from the baseline eGFR value of >20%; and ≥50% decrease in UPCR from baseline, with one of the following conditions met: if the baseline UPCR is ≤3.0 mg/mg, then the post-administration UPCR is <1.0 mg/mg or if the baseline UPCR is >3.0 mg/mg, then the post-administration UPCR is <3.0 mg/mg.

In some embodiments, the LN may be characterized by positive antinuclear antibody (ANA) test results (e.g., ANA titer ≥1:80) in a subject. In some embodiments, the LN may be characterized by elevated anti-dsDNA (e.g., ≥30 IU/mL) or low complement level (e.g., C3<0.9 g/L or C4<0.1 g/L) in a subject. In some embodiments, the LN may be characterized by UPCR ≥1.0 mg/mg in a subject. In some embodiments, the LN may be characterized by eGFR ≥60 mL/min/1.73 m² in a subject. In some embodiments, the LN may be characterized by serum total IgG ≥6 g/L in a subject. In some embodiments, the LN may be characterized by serum total IgG ≥4 g/L in a subject.

In some embodiments, the subject has LN Class III or Class IV. In some embodiments, the subject does not have LN Class III (C), IV-S (C), and IV-G (C). In some embodiments, the subject has LN Class I. In some embodiments, the subject has LN Class II. In some embodiments, the subject has LN Class III. In some embodiments, the subject has LN Class IV. In some embodiments, the subject has LN Class V. In some embodiments, the subject has LN Class VI. In some embodiments, the subject has LN Class III and Class V. In some embodiments, the subject has LN Class IV and Class V.

In some embodiments, the FcRn antagonist is administered at a fixed dose of about 20 mg to about 20,000 mg. In some embodiments, the FcRn antagonist is administered at a fixed dose of about 200 mg to about 20,000 mg. In some embodiments, the FcRn antagonist is administered at a fixed dose of about 300 mg to about 6000 mg. In some embodiments, the FcRn antagonist is administered at a fixed dose of about 750 mg to about 3000 mg. In some embodiments, the FcRn antagonist is administered at a fixed dose of about 1000 mg to about 2500 mg. In some embodiments, the FcRn antagonist is administered at a fixed dose of about 1000 mg to about 2000 mg. In some embodiments, the FcRn antagonist is efgartigimod.

In some embodiments, the FcRn antagonist is administered at a fixed dose of 20 mg to 20,000 mg. In some embodiments, the FcRn antagonist is administered at a fixed dose of 200 mg to 20,000 mg. In some embodiments, the FcRn antagonist is administered at a fixed dose of 300 mg to 6000 mg. In some embodiments, the FcRn antagonist is administered at a fixed dose of 750 mg to 3000 mg. In some embodiments, the FcRn antagonist is administered at a fixed dose of 1000 mg to 2500 mg. In some embodiments, the FcRn antagonist is administered at a fixed dose of 1000 mg to 2000 mg. In some embodiments, the FcRn antagonist is efgartigimod.

In some embodiments, the FcRn antagonist is administered at a fixed dose of about 20 mg, about 50 mg, about 100 mg, about 200 mg, about 250 mg, about 300 mg, about 500 mg, about 750 mg, about 1000 mg, about 1500 mg, about 2000 mg, about 2500 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg. In some embodiments, the FcRn antagonist is efgartigimod.

In some embodiments, the FcRn antagonist is administered at a fixed dose of 20 mg, 50 mg, 100 mg, 200 mg, 250 mg, 300 mg, 500 mg, 750 mg, 1000 mg, 1500 mg, 2000 mg, 2500 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg. In some embodiments, the FcRn antagonist is efgartigimod.

In some embodiments, the FcRn antagonist is administered at a dose of about 0.2 mg/kg to about 200 mg/kg. In some embodiments, the FcRn antagonist is administered at a dose of about 2 mg/kg to about 200 mg/kg. In some embodiments, the FcRn antagonist is administered at a dose of about 2 mg/kg to about 120 mg/kg. In some embodiments, the FcRn antagonist is administered at a dose of about 3 mg/kg to about 60 mg/kg. In some embodiments, the FcRn antagonist is administered at a dose of about 10 mg/kg to about 25 mg/kg. In some embodiments, the FcRn antagonist is efgartigimod.

In some embodiments, the FcRn antagonist is administered at a dose of 0.2 mg/kg to 200 mg/kg. In some embodiments, the FcRn antagonist is administered at a dose of about 2 mg/kg to about 200 mg/kg. In some embodiments, the FcRn antagonist is administered at a dose of 2 mg/kg to 120 mg/kg. In some embodiments, the FcRn antagonist is administered at a dose of 3 mg/kg to 60 mg/kg. In some embodiments, the FcRn antagonist is administered at a dose of 10 mg/kg to 25 mg/kg. In some embodiments, the FcRn antagonist is efgartigimod.

In some embodiments, the FcRn antagonist is administered at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously. In some embodiments, the FcRn antagonist is administered intravenously once weekly, once every two weeks, once every three weeks, once every four weeks, once monthly, or once every six weeks. In some embodiments, the FcRn antagonist is efgartigimod.

In some embodiments, the FcRn antagonist is administered intravenously once weekly or once every two weeks at a dose of about 0.2 mg/kg to about 200 mg/kg. In some embodiments, the FcRn antagonist is administered intravenously once weekly or once every two weeks at a dose of about 2 mg/kg to about 200 mg/kg. In some embodiments, the FcRn antagonist is administered intravenously once weekly or once every two weeks at a dose of about 2 mg/kg to about 120 mg/kg. In some embodiments, the FcRn antagonist is administered intravenously once weekly or once every two weeks at a dose of about 3 mg/kg to about 60 mg/kg. In some embodiments, the FcRn antagonist is administered intravenously once weekly or once every two weeks at a dose of about 10 mg/kg to about 25 mg/kg. In some embodiments, the FcRn antagonist is efgartigimod.

In some embodiments, the FcRn antagonist is administered intravenously once weekly or once every two weeks at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered intravenously once weekly or once every two weeks at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously once weekly or once every two weeks at a dose of about 10 mg/kg to about 30 mg/kg. In some embodiments, the FcRn antagonist is administered intravenously once weekly or once every two weeks at a dose of about 10 mg/kg to about 25 mg/kg. In some embodiments, the FcRn antagonist is administered intravenously once weekly or once every two weeks at a dose of about 10 mg/kg. In some embodiments, the FcRn antagonist is administered intravenously once weekly or once every two weeks at a dose of about 15 mg/kg. In some embodiments, the FcRn antagonist is administered intravenously once weekly or once every two weeks at a dose of about 20 mg/kg. In some embodiments, the FcRn antagonist is administered intravenously once weekly or once every two weeks at a dose of about 25 mg/kg. In some embodiments, the FcRn antagonist is administered intravenously once weekly or once every two weeks at a dose of about 30 mg/kg. In some embodiments, the FcRn antagonist is administered intravenously once weekly or once every two weeks at a dose of 10 mg/kg to 30 mg/kg. In some embodiments, the FcRn antagonist is administered intravenously once weekly or once every two weeks at a dose of 10 mg/kg to 25 mg/kg. In some embodiments, the FcRn antagonist is administered intravenously once weekly or once every two weeks at a dose of 10 mg/kg. In some embodiments, the FcRn antagonist is administered intravenously once weekly or once every two weeks at a dose of 15 mg/kg. In some embodiments, the FcRn antagonist is administered intravenously once weekly or once every two weeks at a dose of 20 mg/kg. In some embodiments, the FcRn antagonist is administered intravenously once weekly or once every two weeks at a dose of 25 mg/kg. In some embodiments, the FcRn antagonist is administered intravenously once weekly or once every two weeks at a dose of 30 mg/kg. In some embodiments, the FcRn antagonist is efgartigimod.

In some embodiments, the FcRn antagonist is administered intravenously once every two weeks for 52 weeks. In some embodiments, the FcRn antagonist is efgartigimod.

In some embodiments, the FcRn antagonist is administered subcutaneously. In some embodiments, the FcRn antagonist is administered subcutaneously once weekly, once every two weeks, once every three weeks, once every four weeks, once monthly, or once every six weeks. In some embodiments, the FcRn antagonist is efgartigimod.

In some embodiments, FcRn antagonist is administered subcutaneously at a fixed dose of about 20 mg to about 20,000 mg. In some embodiments, FcRn antagonist is administered subcutaneously at a fixed dose of about 100 mg to about 10,000 mg once weekly, once every two weeks, once every three weeks, once every four weeks, once monthly, or once every six weeks. In some embodiments, the FcRn antagonist is administered subcutaneously at a fixed dose of 750 mg to 3000 mg once weekly, once every two weeks, once every three weeks, once every four weeks, once monthly, or once every six weeks. In some embodiments, the FcRn antagonist is administered subcutaneously at a fixed dose of 1000 mg to 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of about 20 mg, about 50 mg, about 100 mg, about 250 mg, about 500 mg, about 750 mg, about 1000 mg, about 1500 mg, about 2000 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of 20 mg, 50 mg, 100 mg, 250 mg, 500 mg, 750 mg, 1000 mg, 1500 mg, 2000 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg once weekly, once every two weeks, once every three weeks, once every four weeks, once monthly, or once every six weeks. In some embodiments, the FcRn antagonist is administered subcutaneously at a fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously once weekly or every two weeks at a fixed dose of about 750 mg to about 3000 mg. In some embodiments, the FcRn antagonist is administered subcutaneously once weekly or every two weeks at a fixed dose of about 1000 mg to about 2000 mg. In some embodiments, the FcRn antagonist is administered subcutaneously at a fixed dose of about 1000 mg or about 2000 mg once weekly or every two weeks. In some embodiments, the FcRn antagonist is efgartigimod.

In some embodiments, the FcRn antagonist is administered subcutaneously once weekly or every two weeks at a fixed dose of 750 mg to 3000 mg. In some embodiments, the FcRn antagonist is administered subcutaneously at a fixed dose of 750 mg to 3000 mg once weekly. In some embodiments, the FcRn antagonist is administered subcutaneously at a fixed dose of 750 mg to 3000 mg once every two weeks. In some embodiments, the FcRn antagonist is administered subcutaneously at a fixed dose of 750 mg to 3000 mg once every three weeks. In some embodiments, the FcRn antagonist is administered subcutaneously at a fixed dose of 750 mg to 3000 mg once monthly. In some embodiments, the FcRn antagonist is administered subcutaneously once weekly or every two weeks at a fixed dose of 1000 mg to 2000 mg. In some embodiments, the FcRn antagonist is administered subcutaneously at a fixed dose of 1000 mg or 2000 mg once weekly or every two weeks. In some embodiments, the FcRn antagonist is efgartigimod.

In some embodiments, the FcRn antagonist is first administered subcutaneously at a fixed dose of about 1000 mg twice on the same day. In some embodiments, the FcRn antagonist is first administered subcutaneously at a fixed dose of 1000 mg twice on the same day. In some embodiments, the FcRn antagonist is efgartigimod.

In some embodiments, the FcRn antagonist is administered subcutaneously once weekly at a fixed dose of about 750 mg to about 1750 mg. In some embodiments, the FcRn antagonist is administered subcutaneously once weekly at a fixed dose of about 800 mg to about 1200 mg. In some embodiments, the FcRn antagonist is administered subcutaneously once weekly at a fixed dose of about 750 mg. In some embodiments, the FcRn antagonist is administered subcutaneously once weekly at a fixed dose of about 800 mg. In some embodiments, the FcRn antagonist is administered subcutaneously once weekly at a fixed dose of about 1000 mg. In some embodiments, the FcRn antagonist is administered subcutaneously once weekly at a fixed dose of about 1200 mg. In some embodiments, the FcRn antagonist is administered subcutaneously once weekly at a fixed dose of about 1250 mg. In some embodiments, the FcRn antagonist is administered subcutaneously once weekly at a fixed dose of about 1500 mg. In some embodiments, the FcRn antagonist is administered subcutaneously once weekly at a fixed dose of about 1750 mg. In some embodiments, the FcRn antagonist is efgartigimod.

In some embodiments, the FcRn antagonist is administered subcutaneously once weekly at a fixed dose of 750 mg to 1750 mg. In some embodiments, the FcRn antagonist is administered subcutaneously once weekly at a fixed dose of 800 mg to 1200 mg. In some embodiments, the FcRn antagonist is administered subcutaneously once weekly at a fixed dose of 750 mg. In some embodiments, the FcRn antagonist is administered subcutaneously once weekly at a fixed dose of 800 mg. In some embodiments, the FcRn antagonist is administered subcutaneously once weekly at a fixed dose of 1000 mg. In some embodiments, the FcRn antagonist is administered subcutaneously once weekly at a fixed dose of 1200 mg. In some embodiments, the FcRn antagonist is administered subcutaneously once weekly at a fixed dose of 1250 mg. In some embodiments, the FcRn antagonist is administered subcutaneously once weekly at a fixed dose of 1500 mg. In some embodiments, the FcRn antagonist is administered subcutaneously once weekly at a fixed dose of 1750 mg. In some embodiments, the FcRn antagonist is efgartigimod.

In some embodiments, the FcRn antagonist is administered subcutaneously once weekly at a dose of about 10 mg/kg to about 25 mg/kg. In some embodiments, the FcRn antagonist is administered subcutaneously once weekly at a dose of about 10 mg/kg. In some embodiments, the FcRn antagonist is administered subcutaneously once weekly at a dose of about 15 mg/kg. In some embodiments, the FcRn antagonist is administered subcutaneously once weekly at a dose of about 20 mg/kg. In some embodiments, the FcRn antagonist is administered subcutaneously once weekly at a dose of about 25 mg/kg. In some embodiments, the FcRn antagonist is efgartigimod.

In some embodiments, the FcRn antagonist is administered subcutaneously once weekly at a dose of 10 mg/kg to 25 mg/kg. In some embodiments, the FcRn antagonist is administered subcutaneously once weekly at a dose of 10 mg/kg. In some embodiments, the FcRn antagonist is administered subcutaneously once weekly at a dose of 15 mg/kg. In some embodiments, the FcRn antagonist is administered subcutaneously once weekly at a dose of 20 mg/kg. In some embodiments, the FcRn antagonist is administered subcutaneously once weekly at a dose of 25 mg/kg. In some embodiments, the FcRn antagonist is efgartigimod.

In some embodiments, the FcRn antagonist is first administered intravenously and is subsequently administered subcutaneously. In some embodiments, the FcRn antagonist is first administered intravenously and is subsequently administered subcutaneously at fixed dose of 100 mg to 10,000 mg once weekly, once every two weeks, once every three weeks, once every four weeks, once monthly, or once every six weeks. In some embodiments, the FcRn antagonist is first administered intravenously and is subsequently administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, one or more doses of the FcRn antagonist are administered intravenously and subsequent doses of the FcRn antagonist are administered subcutaneously. In some embodiments, one or more doses of the FcRn antagonist are administered intravenously and subsequent doses of the FcRn antagonist are administered subcutaneously at fixed dose of 100 mg to 10,000 mg once weekly, once every two weeks, once every three weeks, once every four weeks, once monthly, or once every six weeks. In some embodiments, one or more doses of the FcRn antagonist are administered intravenously and subsequent doses of the FcRn antagonist are administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is administered for 6, 12, 24, 39, or 52 weeks or less. In some embodiments, the FcRn antagonist is administered for 24 weeks or less. In some embodiments, the FcRn antagonist is administered for 52 weeks or less. In some embodiments, the FcRn antagonist is administered for at least 6, 12, 24, 39, or 52 weeks. In some embodiments, the FcRn antagonist is administered for at least 24 weeks. In some embodiments, the FcRn antagonist is administered for at least 52 weeks.

In some embodiments, the FcRn antagonist is rozanolixizumab. In some embodiments, rozanolixizumab is administered subcutaneously or intravenously. In some embodiments, rozanolixizumab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, rozanolixizumab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is nipocalimab. In some embodiments, nipocalimab is administered subcutaneously or intravenously. In some embodiments, nipocalimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, nipocalimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is orilanolimab. In some embodiments, orilanolimab is administered subcutaneously or intravenously. In some embodiments, orilanolimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, orilanolimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered intravenously at a dose of about 30 mg/kg once weekly for three weeks and then at a dose of 10 mg/kg administered intravenously every other week.

In some embodiments, the FcRn antagonist is batoclimab. In some embodiments, batoclimab is administered subcutaneously or intravenously. In some embodiments, batoclimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, batoclimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the method further comprises administering to the subject a compound indicated as standard-of-care for LN. In some embodiments, the method further comprises administering to the subject an effective amount of one or more of the following compounds: a glucocorticoid, mycophenolate mofetil (MMF), mycophenolic acid (MPA), an angiotensin-converting enzyme inhibitor (ACEi), an angiotensin receptor blocker (ARB), and/or hydroxychloroquine.

In an embodiment, the method further comprises administering to the subject an effective amount of a corticosteroid and/or an immunosuppressive agent. In an embodiment, the method further comprises administering to the subject an effective amount of a corticosteroid. In an embodiment, method further comprises administering to the subject an effective amount of a glucocorticoid. In an embodiment, the method further comprises administering to the subject an effective amount of a glucocorticoid intravenously and/or administering to the subject an effective amount of a glucocorticoid orally. In an embodiment, the method further comprises administering to the subject an effective amount of a glucocorticoid intravenously and administering to the subject an effective amount of a glucocorticoid orally.

In an embodiment, the method further comprises administering to the subject an effective amount of prednisone. In an embodiment, the method further comprises administering to the subject prednisone at a dose of 7.5 mg/day to 75 mg/day, to a maximum of 1 mg/kg/day. In an embodiment, the method further comprises administering to the subject prednisone at a dose of 8 mg/day to 72 mg/day, to a maximum of 1 mg/kg/day. In an embodiment, the method further comprises administering to the subject prednisone at a dose of 9 mg/day to 66 mg/day, to a maximum of 1 mg/kg/day. In an embodiment, the method further comprises administering to the subject prednisone at a dose of 10 mg/day to 60 mg/day, to a maximum of 1 mg/kg/day. In an embodiment, the method further comprises administering to the subject prednisone at a dose of 0.5 mg/kg/day to 1 mg/kg/day. In an embodiment, the method further comprises administering to the subject prednisone at a dose of 0.6 mg/kg/day to 1 mg/kg/day. In an embodiment, the method further comprises administering to the subject prednisone at a dose of 0.6 mg/kg/day to 1 mg/kg/day, to a maximum of 80 mg/day. In an embodiment, the prednisone is administered orally.

In an embodiment, the method further comprises administering to the subject an effective amount of methylprednisolone. In an embodiment, the method further comprises administering to the subject methylprednisolone at a dose of 100 mg to 1250 mg, for up to three days. In an embodiment, the method further comprises administering to the subject methylprednisolone at a dose of 150 mg to 1200 mg, for up to three days. In an embodiment, the method further comprises administering to the subject methylprednisolone at a dose of 200 mg to 1100 mg, for up to three days. In an embodiment, the method further comprises administering to the subject methylprednisolone at a dose of 500 mg to 1000 mg, for up to three days. In an embodiment, the method further comprises administering to the subject methylprednisolone at a dose of 0.25 g/day to 0.5 g/day. In an embodiment, the method further comprises administering to the subject methylprednisolone at a dose of 0.25 g/day to 0.5 g/day, for one to three days. In an embodiment, the methylprednisolone is administered intravenously.

In an embodiment, the method further comprises administering to the subject an effective amount of prednisone orally and an effective amount of methylprednisolone intravenously. In an embodiment, the method further comprises administering to the subject prednisone orally at a dose of 10 mg/day to 60 mg/day, to a maximum of 1 mg/kg/day, and methylprednisolone intravenously at a dose of 500 mg to 1000 mg, for up to three days. In an embodiment, the method further comprises administering to the subject prednisone orally at a dose of 0.5 mg/kg/day to 1 mg/kg/day, and methylprednisolone intravenously at a dose of 500 mg to 1000 mg, for up to three days. In an embodiment, the method further comprises administering to the subject prednisone orally at a dose of 0.6 mg/kg/day to 1 mg/kg/day, to a maximum of 80 mg/day, and methylprednisolone intravenously at a dose of 0.25 g/day to 0.5 g/day, for one to three days.

In an embodiment, the method further comprises administering to the subject an effective amount of cyclophosphamide. In an embodiment, the method further comprises administering to the subject an effective amount of cyclophosphamide for up to six months. In an embodiment, the method further comprises administering to the subject cyclophosphamide intravenously at a dose of 500 mg once every 2 weeks. In an embodiment, the method further comprises administering to the subject cyclophosphamide intravenously at a dose of 500 mg once every 2 weeks for six months. In an embodiment, the method further comprises administering to the subject cyclophosphamide orally at a dose of 0.5 g/m² to 1 g/m² once a month. In an embodiment, the method further comprises administering to the subject cyclophosphamide orally at a dose of 0.5 g/m² to 1 g/m² once a month for six months.

In an embodiment, the method further comprises administering to the subject an effective amount of a calcineurin inhibitor. In an embodiment, the method further comprises administering to the subject an effective amount of tacrolimus. In an embodiment, the method further comprises administering to the subject tacrolimus, where the trough level is approximately 5.5 ng/mL (6.8 nmol/L). In an embodiment, the method further comprises administering to the subject tacrolimus, where the trough level is approximately 5.5 ng/mL (6.8 nmol/L), and a mycophenolic acid analog. In an embodiment, the method further comprises administering to the subject tacrolimus, where the trough level is approximately 5.5 ng/mL (6.8 nmol/L), and a mycophenolic acid analog, for 24 months. In an embodiment, in a subject with a serum creatinine (SCr) level of less than 3 mg/dL (265 μmol/L), the method further comprises administering to the subject tacrolimus, where the trough level is approximately 5.5 ng/mL (6.8 nmol/L), and a mycophenolic acid analog, for 24 months. In an embodiment, the method further comprises administering to the subject an effective amount of voclosporin. In an embodiment, the method further comprises administering to the subject voclosporin at a dose of 23.7 mg twice a day. In an embodiment, the method further comprises administering to the subject voclosporin at a dose of 23.7 mg twice a day, and a mycophenolic acid analog. In an embodiment, the method further comprises administering to the subject voclosporin at a dose of 23.7 mg twice a day, and a mycophenolic acid analog, for 52 weeks. In an embodiment, in a subject with an eGFR of greater than 45 mL/min per 1.73 m², the method further comprises administering to the subject voclosporin at a dose of 23.7 mg twice a day, and a mycophenolic acid analog, for 52 weeks.

In an embodiment, when the subject is administered the FcRn antagonist, the dose of prednisone is reduced over 12 weeks to a dose of 7.5 mg/day.

In an embodiment, the method further comprises administering to the subject an effective amount of mycophenolate mofetil (MMF), mycophenolic acid (MPA), or an analog thereof. In an embodiment, the method further comprises administering to the subject a daily dose of MMF, MPA, or an analog thereof. In an embodiment, the dose of MMF or MPA is increased to a maximum of 1.5 g or 2 g/day, or equivalent, in divided doses within four weeks. In an embodiment, the method further comprises administering to the subject MMF orally at a dose of 1 g to 1.5 g twice a day. In an embodiment, the method further comprises administering to the subject mycophenolic acid sodium orally at a dose of 0.72 g to 1.08 g twice a day.

In an embodiment, the method further comprises administering to the subject an effective amount of a B-lymphocyte targeting biologic. Examples of B-lymphocyte targeting biologics include, without limitation, belimumab, rituximab, and obinutuzumab. In an embodiment, the method further comprises administering to the subject an effective amount of belimumab. In an embodiment, the method further comprises administering to the subject belimumab intravenously at a dose of 10 mg/kg once every two weeks for three doses, then once every four weeks for subsequent doses. In an embodiment, the method further comprises administering to the subject belimumab intravenously at a dose of 10 mg/kg once every two weeks for three doses, then once every four weeks for subsequent doses, and a mycophenolic acid analog. In an embodiment, the method further comprises administering to the subject belimumab intravenously at a dose of 10 mg/kg once every two weeks for three doses, then once every four weeks for subsequent doses, and cyclophosphamide. In an embodiment, the method further comprises administering to the subject belimumab intravenously at a dose of 10 mg/kg once every two weeks for three doses, then once every four weeks for subsequent doses, and cyclophosphamide at a dose of 500 mg once every two weeks for six months. In an embodiment, the method further comprises administering to the subject an effective amount of rituximab. In an embodiment, the method further comprises administering to the subject rituximab at a dose of 1 g on days 1 and 15 as add-on therapy for refractory cases or for corticosteroid minimization. In an embodiment, the method further comprises administering to the subject an effective amount of obinutuzumab.

In some embodiments, the FcRn antagonist is administered intravenously once every two weeks, together with MMF or MPA and glucocorticoid treatment, for 52 weeks. In some embodiments, the FcRn antagonist is efgartigimod.

In an embodiment, the method further comprises administering to the subject an effective amount of an ACEi or ARB.

In an embodiment, the method further comprises administering to the subject an effective amount of hydroxychloroquine. In some embodiments, the maximum dose of hydroxychloroquine is 5 mg/kg/day. In an embodiment, the hydroxychloroquine is administered for 24 weeks. In an embodiment, the hydroxychloroquine is administered for at least 24 weeks.

In an embodiment, the subject was previously treated with an induction therapy for LN. In an embodiment, the subject has received an induction therapy for LN. In an embodiment, the induction therapy is initiated within 60 days prior to administering the FcRn antagonist to the subject. In an embodiment, the induction therapy is initiated less than 60 days prior to administering the FcRn antagonist to the subject. In an embodiment, the induction therapy is initiated at least three days, but no more than 60 days (e.g., three days, four days, 5 days, 10 days, 15 days, 20 days, 25 days, 30 days, 35 days, 40 days, 45 days, 50 days, 55 days, 60 days) prior to administering the FcRn antagonist to the subject. In an embodiment, the subject has received the induction therapy within 60 days prior to administering the FcRn antagonist to the subject. In an embodiment, the method comprises administering to the subject an effective amount of a human FcRn antagonist after the subject has received an induction therapy for LN. In an embodiment, the method comprises administering to the subject an effective amount of a human FcRn antagonist within 60 days of the subject receiving an induction therapy for LN.

In an embodiment, the subject was previously treated with an effective amount of a corticosteroid and/or an immunosuppressive agent. In an embodiment, the subject was previously treated with prednisone. In an embodiment, the induction therapy comprises administering to the subject an effective amount of a corticosteroid and/or an immunosuppressive agent. In an embodiment, the induction therapy comprises administering to the subject an effective amount of a corticosteroid. In an embodiment, the induction therapy comprises administering to the subject an effective amount of a glucocorticoid. In an embodiment, the induction therapy comprises administering to the subject an effective amount of a glucocorticoid intravenously and/or administering to the subject a glucocorticoid orally. In an embodiment, the induction therapy comprises administering to the subject an effective amount of a glucocorticoid intravenously and administering to the subject a glucocorticoid orally.

In an embodiment, the subject was previously treated with an effective amount of prednisone. In an embodiment, the subject was previously treated with prednisone at a dose of 7.5 mg/day to 75 mg/day, to a maximum of 1 mg/kg/day. In an embodiment, the subject was previously treated with prednisone at a dose of 8 mg/day to 72 mg/day, to a maximum of 1 mg/kg/day. In an embodiment, the subject was previously treated with prednisone at a dose of 9 mg/day to 66 mg/day, to a maximum of 1 mg/kg/day. In an embodiment, the subject was previously treated with prednisone at a dose of 10 mg/day to 60 mg/day, to a maximum of 1 mg/kg/day. In an embodiment, the subject was previously treated with prednisone at a dose of 0.6 mg/kg/day to 1 mg/kg/day. In an embodiment, the method further comprises administering to the subject prednisone at a dose of 0.6 mg/kg/day to 1 mg/kg/day, to a maximum of 80 mg/day. In an embodiment, the induction therapy comprises administering to the subject an effective amount of prednisone. In an embodiment, the induction therapy comprises administering to the subject prednisone at a dose of 7.5 mg/day to 75 mg/day, to a maximum of 1 mg/kg/day. In an embodiment, the induction therapy comprises administering to the subject prednisone at a dose of 8 mg/day to 72 mg/day, to a maximum of 1 mg/kg/day. In an embodiment, the induction therapy comprises administering to the subject prednisone at a dose of 9 mg/day to 66 mg/day, to a maximum of 1 mg/kg/day. In an embodiment, the induction therapy comprises administering to the subject prednisone at a dose of 10 mg/day to 60 mg/day, to a maximum of 1 mg/kg/day. In an embodiment, the induction therapy comprises administering to the subject prednisone at a dose of 0.5 mg/kg/day to 1 mg/kg/day. In an embodiment, the induction therapy comprises administering to the subject prednisone at a dose of 0.6 mg/kg/day to 1 mg/kg/day. In an embodiment, the method further comprises administering to the subject prednisone at a dose of 0.6 mg/kg/day to 1 mg/kg/day, to a maximum of 80 mg/day. In an embodiment, the prednisone is administered orally.

In an embodiment, the subject was previously treated with an effective amount of methylprednisolone. In an embodiment, the subject was previously treated with methylprednisolone at a dose of 100 mg to 1250 mg, for up to three days. In an embodiment, the subject was previously treated with methylprednisolone at a dose of 150 mg to 1200 mg, for up to three days. In an embodiment, the subject was previously treated with methylprednisolone at a dose of 200 mg to 1100 mg, for up to three days. In an embodiment, the subject was previously treated with methylprednisolone at a dose of 500 mg to 1000 mg, for up to three days. In an embodiment, the subject was previously treated with methylprednisolone at a dose of 0.25 g/day to 0.5 g/day. In an embodiment, the method further comprises administering to the subject methylprednisolone at a dose of 0.25 g/day to 0.5 g/day, for one to three days. In an embodiment, the induction therapy comprises administering to the subject an effective amount of methylprednisolone. In an embodiment, the induction therapy comprises administering to the subject methylprednisolone at a dose of 100 mg to 1250 mg, for up to three days. In an embodiment, the induction therapy comprises administering to the subject methylprednisolone at a dose of 150 mg to 1200 mg, for up to three days. In an embodiment, the induction therapy comprises administering to the subject methylprednisolone at a dose of 200 mg to 1100 mg, for up to three days. In an embodiment, the induction therapy comprises administering to the subject methylprednisolone at a dose of 500 mg to 1000 mg, for up to three days. In an embodiment, the induction therapy comprises administering to the subject methylprednisolone at a dose of 0.25 g/day to 0.5 g/day. In an embodiment, the method further comprises administering to the subject methylprednisolone at a dose of 0.25 g/day to 0.5 g/day, for one to three days. In an embodiment, the methylprednisolone is administered intravenously.

In an embodiment, the subject was previously treated with an effective amount of prednisone orally and methylprednisolone intravenously. In an embodiment, the subject was previously treated with prednisone orally at a dose of 10 mg/day to 60 mg/day, to a maximum of 1 mg/kg/day and methylprednisolone intravenously at a dose of 500 mg to 1000 mg, for up to three days. In an embodiment, the induction therapy comprises administering to the subject prednisone orally and methylprednisolone intravenously. In an embodiment, the subject was previously treated with prednisone orally at a dose of 0.6 mg/kg/day to 1 mg/kg/day, to a maximum of 80 mg/day, and methylprednisolone intravenously at a dose of 0.25 g/day to 0.5 g/day, for one to three days. In an embodiment, the induction therapy comprises administering to the subject an effective amount of prednisone orally at a dose of 10 mg/day to 60 mg/day, to a maximum of 1 mg/kg/day and methylprednisolone intravenously at a dose of 500 mg to 1000 mg, for up to three days. In an embodiment, the induction therapy comprises administering to the subject prednisone orally at a dose of 0.5 mg/kg/day to 1 mg/kg/day and methylprednisolone intravenously at a dose of 500 mg to 1000 mg, for up to three days. In an embodiment, the induction therapy comprises administering to the subject prednisone orally at a dose of 0.6 mg/kg/day to 1 mg/kg/day, to a maximum of 80 mg/day, and methylprednisolone intravenously at a dose of 0.25 g/day to 0.5 g/day, for one to three days.

In an embodiment, the subject was previously treated with an effective amount of cyclophosphamide. In an embodiment, the subject was previously treated with an effective amount of cyclophosphamide for up to six months. In an embodiment, the subject was previously treated with cyclophosphamide intravenously at a dose of 500 mg once every 2 weeks. In an embodiment, the subject was previously treated with cyclophosphamide intravenously at a dose of 500 mg once every 2 weeks for six months. In an embodiment, the subject was previously treated with cyclophosphamide orally at a dose of 0.5 g/m² to 1 g/m² once a month. In an embodiment, the subject was previously treated with cyclophosphamide orally at a dose of 0.5 g/m² to 1 g/m² once a month for six months. In an embodiment, the induction therapy comprises administering to the subject an effective amount of cyclophosphamide. In an embodiment, the induction therapy comprises administering to the subject an effective amount of cyclophosphamide for up to six months. In an embodiment, the induction therapy comprises administering to the subject cyclophosphamide intravenously at a dose of 500 mg once every 2 weeks. In an embodiment, the induction therapy comprises administering to the subject cyclophosphamide intravenously at a dose of 500 mg once every 2 weeks for six months. In an embodiment, the induction therapy comprises administering to the subject cyclophosphamide orally at a dose of 0.5 g/m² to 1 g/m² once a month. In an embodiment, the induction therapy comprises administering to the subject cyclophosphamide orally at a dose of 0.5 g/m² to 1 g/m² once a month for six months.

In an embodiment, the subject was previously treated with an effective amount of a calcineurin inhibitor. In an embodiment, the method further comprises administering to the subject an effective amount of tacrolimus. In an embodiment, the subject was previously treated with tacrolimus, where the trough level is approximately 5.5 ng/mL (6.8 nmol/L). In an embodiment, the subject was previously treated with tacrolimus, where the trough level is approximately 5.5 ng/mL (6.8 nmol/L), and a mycophenolic acid analog. In an embodiment, the subject was previously treated with tacrolimus, where the trough level is approximately 5.5 ng/mL (6.8 nmol/L), and a mycophenolic acid analog, for 24 months. In an embodiment, in a subject with a SCr level of less than 3 mg/dL (265 μmol/L), the subject was previously treated with tacrolimus, where the trough level is approximately 5.5 ng/mL (6.8 nmol/L), and a mycophenolic acid analog, for 24 months. In an embodiment, the subject was previously treated with an effective amount of voclosporin. In an embodiment, the subject was previously treated with voclosporin at a dose of 23.7 mg twice a day. In an embodiment, the subject was previously treated with voclosporin at a dose of 23.7 mg twice a day, and a mycophenolic acid analog. In an embodiment, the subject was previously treated with voclosporin at a dose of 23.7 mg twice a day, and a mycophenolic acid 5 analog, for 52 weeks. In an embodiment, in a subject with an eGFR of greater than 45 mL/min per 1.73 m², the subject was previously treated with voclosporin at a dose of 23.7 mg twice a day, and a mycophenolic acid analog, for 52 weeks. In an embodiment, the induction therapy comprises administering to the subject an effective amount of a calcineurin inhibitor. In an embodiment, the method further comprises administering to the subject an effective amount of tacrolimus. In an embodiment, the induction therapy comprises administering to the subject tacrolimus, where the trough level is approximately 5.5 ng/mL (6.8 nmol/L). In an embodiment, the induction therapy comprises administering to the subject tacrolimus, where the trough level is approximately 5.5 ng/mL (6.8 nmol/L), and a mycophenolic acid analog. In an embodiment, the induction therapy comprises administering to the subject tacrolimus, where the trough level is approximately 5.5 ng/mL (6.8 nmol/L), and a mycophenolic acid analog, for 24 months. In an embodiment, in a subject with a SCr level of less than 3 mg/dL (265 μmol/L), the induction therapy comprises administering to the subject tacrolimus, where the trough level is approximately 5.5 ng/mL (6.8 nmol/L), and a mycophenolic acid analog, for 24 months. In an embodiment, the induction therapy comprises administering to the subject an effective amount of voclosporin. In an embodiment, the induction therapy comprises administering to the subject voclosporin at a dose of 23.7 mg twice a day. In an embodiment, the induction therapy comprises administering to the subject voclosporin at a dose of 23.7 mg twice a day, and a mycophenolic acid analog. In an embodiment, the induction therapy comprises administering to the subject voclosporin at a dose of 23.7 mg twice a day, and a mycophenolic acid analog, for 52 weeks. In an embodiment, in a subject with an eGFR of greater than 45 mL/min per 1.73 m², the induction therapy comprises administering to the subject voclosporin at a dose of 23.7 mg twice a day, and a mycophenolic acid analog, for 52 weeks.

In an embodiment, when the subject is administered the FcRn antagonist, the dose of prednisone is tapered over 12 weeks to a dose of 7.5 mg/day. In an embodiment, the initial oral prednisone dose is at 0.5 mg/kg/day to 1 mg/kg/day, not to exceed 60 mg/day. In an embodiment, the initial oral prednisone dose is rounded to the nearest 10 mg increment set forth in Table S2, and prednisone taper is initiated 2 weeks after initiation of oral prednisone according to the schedule set forth in Table S2. In some embodiments, for subjects administered the FcRn antagonist who experience an LN flare, subjects may be administered post-LN flare prednisone at a dose of 0.5 mg/kg/day to 1 mg/kg/day, not to exceed 60 mg/day, for 2 weeks. In an embodiment, the post-LN flare prednisone is tapered within 6 weeks to a dose of 10 mg/day or less. In an embodiment, the post-LN flare prednisone is tapered within 6 weeks to a dose of 10 mg/day or less according to the schedule set forth in Table S3. In some embodiments, for subjects administered the FcRn antagonist who experience an extrarenal flare, subjects may be administered post-extrarenal flare prednisone at a dose of up to 1 mg/kg/day, not to exceed 60 mg/day. In an embodiment, the post-extrarenal flare prednisone is tapered over about 12 weeks to a dose of 7.5 mg/day. In an embodiment, the post-extrarenal flare prednisone is tapered over about 12 weeks to a dose of 7.5 mg/day according to the schedule set forth in Table S4.

In an embodiment, the subject was previously treated with an effective amount of MMF, MPA, or an analog thereof. In an embodiment, the induction therapy comprises administering to the subject an effective amount of MMF, MPA, or an analog thereof. In an embodiment, the dose of MMF or MPA is increased to a maximum of 1.5 g or 2 g/day, or equivalent, in divided doses within four weeks. In an embodiment, the subject was previously treated with MMF orally at a dose of 1 g to 1.5 g twice a day. In an embodiment, the subject was previously treated with mycophenolic acid sodium orally at a dose of 0.72 g to 1.08 g twice a day. In an embodiment, the induction therapy comprises administering to the subject MMF orally at a dose of 1 g to 1.5 g twice a day. In an embodiment, the induction therapy comprises administering to the subject mycophenolic acid sodium orally at a dose of 0.72 g to 1.08 g twice a day. In an embodiment, the method further comprises administering to the subject a daily dose of an effective amount of MMF, MPA, or an analog thereof. In an embodiment, the dose of MMF, MPA, or an analog thereof is increased to a maximum of 1.5 g or 2 g/day, or equivalent, in divided doses within four weeks.

In an embodiment, the subject was previously treated with an effective amount of a B-lymphocyte targeting biologic. Examples of B-lymphocyte targeting biologics include, without limitation, belimumab, rituximab, and obinutuzumab. In an embodiment, the subject was previously treated with an effective amount of belimumab. In an embodiment, the subject was previously treated with belimumab intravenously at a dose of 10 mg/kg once every two weeks for three doses, then once every four weeks for subsequent doses. In an embodiment, the subject was previously treated with belimumab intravenously at a dose of 10 mg/kg once every two weeks for three doses, then once every four weeks for subsequent doses, and a mycophenolic acid analog. In an embodiment, the subject was previously treated with belimumab intravenously at a dose of 10 mg/kg once every two weeks for three doses, then once every four weeks for subsequent doses, and cyclophosphamide. In an embodiment, the subject was previously treated with belimumab intravenously at a dose of 10 mg/kg once every two weeks for three doses, then once every four weeks for subsequent doses, and cyclophosphamide at a dose of 500 mg once every two weeks for six months. In an embodiment, the subject was previously treated with an effective amount of rituximab. In an embodiment, the subject was previously treated with rituximab at a dose of 1 g on days 1 and 15 as add-on therapy for refractory cases or for corticosteroid minimization. In an embodiment, the subject was previously treated with an effective amount of obinutuzumab.

In an embodiment, the induction therapy comprises administering to the subject an effective amount of a B-lymphocyte targeting biologic. Examples of B-lymphocyte targeting biologics include, without limitation, belimumab, rituximab, and obinutuzumab. In an embodiment, the induction therapy comprises administering to the subject an effective amount of belimumab. In an embodiment, the induction therapy comprises administering to the subject belimumab intravenously at a dose of 10 mg/kg once every two weeks for three doses, then once every four weeks for subsequent doses. In an embodiment, the induction therapy comprises administering to the subject belimumab intravenously at a dose of 10 mg/kg once every two weeks for three doses, then once every four weeks for subsequent doses, and a mycophenolic acid analog. In an embodiment, the induction therapy comprises administering to the subject belimumab intravenously at a dose of 10 mg/kg once every two weeks for three doses, then once every four weeks for subsequent doses, and cyclophosphamide. In an embodiment, the induction therapy comprises administering to the subject belimumab intravenously at a dose of 10 mg/kg once every two weeks for three doses, then once every four weeks for subsequent doses, and cyclophosphamide at a dose of 500 mg once every two weeks for six months. In an embodiment, the induction therapy comprises administering to the subject an effective amount of rituximab. In an embodiment, the induction therapy comprises administering to the subject rituximab at a dose of 1 g on days 1 and 15 as add-on therapy for refractory cases or for corticosteroid minimization. In an embodiment, the induction therapy comprises administering to the subject an effective amount of obinutuzumab.

In an embodiment, the subject was previously treated with an ACEi or ARB. In an embodiment, the method further comprises administering to the subject an ACEi or ARB.

In an embodiment, the subject was previously treated with hydroxychloroquine at a maximum dose of 5 mg/kg/day. In an embodiment, the method further comprises administering to the subject hydroxychloroquine at a maximum dose of 5 mg/kg/day. In an embodiment, the hydroxychloroquine is administered for 24 weeks. In an embodiment, the hydroxychloroquine is administered for at least 24 weeks.

In some embodiments, treatment of LN is characterized by the subject exhibiting an eGFR of at least 25 mL/min/1.73 m². In some embodiments, the subject exhibits an eGFR of at least 30 mL/min/1.73 m², at least 35 mL/min/1.73 m², at least 40 mL/min/1.73 m², at least 45 mL/min/1.73 m², at least 50 mL/min/1.73 m², at least 55 mL/min/1.73 m², at least 60 mL/min/1.73 m², at least 65 mL/min/1.73 m², at least 70 mL/min/1.73 m², at least 75 mL/min/1.73 m², at least 80 mL/min/1.73 m², at least 85 mL/min/1.73 m², at least 90 mL/min/1.73 m², at least 95 mL/min/1.73 m², at least 100 mL/min/1.73 m², at least 105 mL/min/1.73 m², at least 110 mL/min/1.73 m², at least 115 mL/min/1.73 m², at least 120 mL/min/1.73 m². In some embodiments, the subject exhibits an eGFR of at least 60 mL/min/1.73 m². In some embodiments, treatment of LN is characterized by the subject exhibiting less than 20% reduction in eGFR as compared to a baseline eGFR obtained from the subject prior to administering the FcRn antagonist. In some embodiments, the subject exhibits less than 15%, less than 10%, less than 5%, less than 1% reduction in eGFR as compared to a baseline eGFR obtained from the subject prior to administering the FcRn antagonist.

In some embodiments, after administering the FcRn antagonist to the subject, the subject exhibits a post-administration eGFR of at least 60 mL/min/1.73 m². In some embodiments, after administering the FcRn antagonist to the subject, the subject exhibits a post-administration eGFR that is less than 20% reduced as compared to a baseline eGFR obtained from the subject prior to administering the FcRn antagonist. In some embodiments, the post-administration eGFR is measured 24 weeks after administering the FcRn antagonist to the subject. In some embodiments, the post-administration eGFR is measured 52 weeks after administering the FcRn antagonist to the subject.

In some embodiments, after administering the FcRn antagonist to the subject, the subject exhibits a post-administration eGFR of at least 90 mL/min/1.73 m². In some embodiments, after administering the FcRn antagonist to the subject, the subject exhibits a post-administration eGFR that is less than 10% reduced as compared to a baseline eGFR obtained from the subject prior to administering the FcRn antagonist. In some embodiments, the post-administration eGFR is measured 24 weeks after administering the FcRn antagonist to the subject. In some embodiments, the post-administration eGFR is measured 52 weeks after administering the FcRn antagonist to the subject.

In some embodiments, treatment of LN is characterized by the subject exhibiting a UPCR of at most 0.5 mg/mg. In some embodiments, the subject exhibits a UPCR of at most 0.45 mg/mg, 0.4 mg/mg, 0.35 mg/mg, 0.3 mg/mg, 0.25 mg/mg, 0.2 mg/mg, 0.15 mg/mg, 0.1 mg/mg.

In some embodiments, treatment of LN is characterized by the subject exhibiting at least 50% reduction in UPCR as compared to a baseline UPCR obtained from the subject prior to administering the FcRn antagonist, and if the baseline UPCR is at most 3 mg/mg, then the post-administration UPCR is less than 1 mg/mg. In some embodiments, treatment of LN is characterized by the subject exhibiting at least 50% reduction in UPCR as compared to a baseline UPCR obtained from the subject prior to administering the FcRn antagonist, and if the baseline UPCR is greater than 3 mg/mg, then the post-administration UPCR is less than 3 mg/mg.

In some embodiments, treatment of LN is characterized by the reduction of circulating immune complexes. In some embodiments, the circulating immune complexes are selected from the group consisting of C3, C4, CH50, and C1q-binding circulating immune complexes. In some embodiments, the prevalence of circulating immune complexes is reduced by at least 10%, at least 25%, at least 50%, at least 75%, at least 80%, at least 90%, at least 95%, at least 96%, at least 97%, at least 98%, or at least 99% after administering one or more of the treatments described herein. In some embodiments, circulating immune complexes are undetectable in the subject after administering one or more of the treatments described herein. In some embodiments, the treatment is administration of an effective amount of an FcRn antagonist. In some embodiments, the treatment is administration of an effective amount of efgartigimod.

In some embodiments, after administering the FcRn antagonist to the subject, the subject exhibits a post-administration UPCR of at most 0.5 mg/mg. In some embodiments, after administering the FcRn antagonist to the subject, the subject exhibits a post-administration UPCR that is at least 50% reduced as compared to a baseline UPCR obtained from the subject prior to administering the FcRn antagonist, and wherein if the baseline UPCR is at most 3 mg/mg, then the post-administration UPCR is less than 1 mg/mg. In some embodiments, after administering the FcRn antagonist to the subject, the subject exhibits a post-administration UPCR that is at least 50% reduced as compared to a baseline UPCR obtained from the subject prior to administering the FcRn antagonist, and wherein if the baseline UPCR is greater than 3 mg/mg, then the post-administration UPCR is less than 3 mg/mg. In some embodiments, the post-administration UPCR is measured 24 weeks after administering the FcRn antagonist to the subject. In some embodiments, the post-administration UPCR is measured 52 weeks after administering the FcRn antagonist to the subject.

In some embodiments, after administering the FcRn antagonist to the subject, the subject exhibits a post-administration Systemic Lupus Erythematosus Disease Activity Index (SLEDAI)-2K score that is reduced as compared to a baseline SLEDAI-2K score obtained from the subject prior to administering the FcRn antagonist. In some embodiments, the post-administration SLEDAI-2K score is measured 24 weeks after administering the FcRn antagonist to the subject. In some embodiments, the post-administration SLEDAI-2K score is measured 52 weeks after administering the FcRn antagonist to the subject.

In some embodiments, after administering the FcRn antagonist to the subject, the subject exhibits a post-administration level of a serum autoantibody that is reduced as compared to a baseline level of the serum autoantibody obtained from the subject prior to administering the FcRn antagonist. In some embodiments, the post-administration level of the serum autoantibody is reduced by at least 10%, at least 20%, at least 30%, at least 40%, at least 50%, at least 60%, at least 70%, at least 80%, at least 90%, or 100%, as compared to the baseline level of the serum autoantibody obtained from the subject prior to administering the FcRn antagonist. In some embodiments, the post-administration level of the serum autoantibody is measured 24 weeks after administering the FcRn antagonist to the subject. In some embodiments, the post-administration level of the serum autoantibody is measured 52 weeks after administering the FcRn antagonist to the subject. In some embodiments, the serum autoantibody is selected from the group consisting of anti-dsDNA, ANA, aCL, anti-Sm, and anti-C1q.

In some embodiments, after administering the FcRn antagonist to the subject, the subject exhibits a post-administration level of a serum complement that is reduced as compared to a baseline level of the serum complement obtained from the subject prior to administering the FcRn antagonist. In some embodiments, the post-administration level of the serum complement is reduced by at least 10%, at least 20%, at least 30%, at least 40%, at least 50%, at least 60%, at least 70%, at least 80%, at least 90%, or 100%, as compared to the baseline level of the serum complement obtained from the subject prior to administering the FcRn antagonist. In some embodiments, the post-administration level of the serum complement is measured 24 weeks after administering the FcRn antagonist to the subject. In some embodiments, the post-administration level of the serum complement is measured 52 weeks after administering the FcRn antagonist to the subject. In some embodiments, the serum complement is selected from the group consisting of C3, C4, CH50, and C1q-binding circulating immune complexes.

In some embodiments, after administering the FcRn antagonist to the subject, the subject exhibits a post-administration level of circulating immune complexes that is reduced as compared to a baseline level of circulating immune complexes obtained from the subject prior to administering the FcRn antagonist. In some embodiments, the circulating immune complexes are selected from the group consisting of C3, C4, CH50, and C1q-binding circulating immune complexes. In some embodiments, the post-administration level of circulating immune complexes is reduced by at least 10%, at least 25%, at least 50%, at least 75%, at least 80%, at least 90%, at least 95%, at least 96%, at least 97%, at least 98%, or at least 99%, as compared to the baseline level of circulating immune complexes obtained from the subject prior to administering the FcRn antagonist. In some embodiments, the post-administration level of circulating immune complexes is measured 24 weeks after administering the FcRn antagonist to the subject. In some embodiments, the post-administration level of circulating immune complexes is measured 52 weeks after administering the FcRn antagonist to the subject.

In some embodiments, after administering the FcRn antagonist to the subject, the subject exhibits a post-administration level of serum IgG that is reduced as compared to a baseline level of serum IgG obtained from the subject prior to administering the FcRn antagonist. In some embodiments, the post-administration level of serum IgG is reduced by at least 10%, at least 20%, at least 30%, at least 40%, at least 50%, at least 60%, at least 70%, at least 80%, at least 90%, or 100%, as compared to the baseline level of serum IgG obtained from the subject prior to administering the FcRn antagonist. In some embodiments, the post-administration level of serum IgG is measured 24 weeks after administering the FcRn antagonist to the subject. In some embodiments, the post-administration level of serum IgG is measured 52 weeks after administering the FcRn antagonist to the subject.

In some embodiments, after administering the FcRn antagonist to the subject, the level of albumin is not decreased in the subject following administration of the FcRn antagonist compared to a baseline level of albumin. In some embodiments, after administering the FcRn antagonist to the subject, the subject exhibits a post-administration level of albumin that is not decreased as compared to a baseline level of albumin obtained from the subject prior to administering the FcRn antagonist. In an embodiment, an albumin reduction of less than about 1%, 2%, 3%, 4%, or 5% compared to baseline albumin level is observed. In an embodiment, an albumin reduction of less than about 10% compared to baseline albumin level is observed. In some embodiments, the post-administration level of albumin is measured 24 weeks after administering the FcRn antagonist to the subject. In some embodiments, the post-administration level of albumin is measured 52 weeks after administering the FcRn antagonist to the subject.

In some embodiments, after administering the FcRn antagonist to the subject, the level of serum albumin is not reduced in the subject following administration of the FcRn antagonist compared to a baseline level of serum albumin. In some embodiments, after administering the FcRn antagonist to the subject, the subject exhibits a post-administration level of serum albumin that is not reduced as compared to a baseline level of serum albumin obtained from the subject prior to administering the FcRn antagonist. In an embodiment, a serum albumin reduction of less than about 1%, 2%, 3%, 4%, or 5% compared to baseline serum albumin level is observed. In an embodiment, a serum albumin reduction of less than about 10% compared to baseline serum albumin level is observed. In some embodiments, the post-administration level of serum albumin is measured 24 weeks after administering the FcRn antagonist to the subject. In some embodiments, the post-administration level of serum albumin is measured 52 weeks after administering the FcRn antagonist to the subject.

In an embodiment, the subject is any human or non-human animal. In an embodiment, the subject is a human or non-human mammal. In an embodiment, the subject is a human. In an embodiment, the subject is of Asian descent.

### EXAMPLES

The following examples are offered by way of illustration, and not by way of limitation.

### Example 1: Investigation of Efficacy of Efgartigimod on Reducing Circulating Immune Complexes

Immunocomplexes are found in healthy individuals, but their formation can be expected to be elevated in autoimmune diseases and partly drive and/or exaggerate their pathologies. Efgartigimod is an engineered Fc fragment that inhibits FcRn function by outcompeting endogenous IgG binding, resulting in reduced IgG recycling and increased IgG degradation. As the half-life of immune complexes, largely containing IgG, may also be affected by the biology of FcRn, circulating immune complex (CIC) levels were investigated as part of an open-label, adaptive phase 2 trial of extended efgartigimod treatment in participants with mild to moderate pemphigus vulgaris (PV) or pemphigus foliaceus (PF) (ClinicalTrials.gov: NCT03334058; Maho-Vaillant M et al. Front Immunol (2022) 13:863095). Briefly, seven participants (7 PV) were treated for 15 weeks in cohort 3 and 15 participants (8 PV, 7 PF) were treated up to 34 weeks in cohort 4 as reported earlier (Goebeler M et al. Br J Dermatol (2021) 186(3):429-39). PV or PF diagnosis was confirmed by positive direct immunofluorescence and positive indirect immunofluorescence and/or Dsg-1/3 ELISA. Participants were either newly diagnosed or relapsing, with mild to moderate disease severity (pemphigus disease area index [PDAI]<45 at baseline). The treatment period was preceded by a screening period of up to 3 weeks and followed by a treatment-free follow-up period of 10 weeks. In cohort 3, intravenous (IV) efgartigimod was administered weekly at 10 mg/kg for 4 weeks as induction followed by administrations every other week for 12 weeks as maintenance period. In cohort 3, efgartigimod could be initiated as monotherapy or in combination with 20 mg/day of prednisone at the discretion of the investigator, and prednisone could be tapered starting in the maintenance period. In cohort 4, IV efgartigimod was dosed weekly at 25 mg/kg body weight until EoC, defined as the time at which no new lesions have developed for a minimum of 2 weeks and the majority, i.e., approximately 80% of established lesions have healed. Thereafter, participants were dosed every other week. In cohort 4, efgartigimod was initiated with concomitant prednisone (20 mg/day) in all newly diagnosed participants and relapsing participants off therapy, or at the tapered dose at which relapse occurred. The oral prednisone dose could be tapered as of EoC. No other systemic treatments for pemphigus were permitted during the study, but topical corticosteroids, analgesics, and supportive care for corticosteroid therapy (e.g., vitamin D, proton-pump inhibitors, and specific diets) were allowed.

Suppression of CIC was measured in six participants with PV and six participants with PF who received extended efgartigimod treatment in cohorts 3 and 4 and achieved a sustained clinical response. These participants were subjected to IgG CIC analysis by C1q ELISA, which detects complement-fixed IgG antibodies (without providing information about antigen specificity of CICs). Briefly, CIC-C1q EIA kit (A001, Quidel) was employed to detect levels of C1q-associated IgG aggregates in sera of the selected participants at different time points according to the manufacturer's protocol. Calibrators provided by the kit were used to determine expression level. IgG CIC levels are considered clinically significant if ≥4.0 mg Eq/ml.

Four participants presented with elevated baseline CIC levels, but notable reduction of CICs during efgartigimod treatment was observed (FIG. 1), consistent with the observed improvement in their clinical condition. This demonstrates that efgartigimod treatment reduced CIC in pemphigus patients.

### Example 2: Investigation of Efficacy and Safety of Efgartigimod in Chinese Patients with Lupus Nephritis (LN)

Systemic lupus erythematosus (SLE) is a chronic heterogenous autoimmune disease. LN is an inflammatory autoimmune disease of the kidney caused by SLE and is the most common life-threatening manifestation of SLE.

In unselected patients with SLE, approximately 25% to 50% have signs or symptoms of kidney disease at SLE onset, and approximately 40% to 60% of patients with SLE will develop renal involvement during the course of the disease. In China, approximately half of the patients with SLE have renal involvement.

In active proliferative LN, induction treatment with mycophenolate mofetil (MMF) or mycophenolic acid (MPA), or low-dose intravenous (IV) cyclophosphamide (CYC), both combined with glucocorticoids, is currently recommended. Alternative regimens include treatment with MMF and calcineurin inhibitors (e.g., tacrolimus), and high-dose CYC. Subsequent maintenance therapy includes treatment with MMF or azathioprine with no or low-dose glucocorticoids. Despite aggressive immunosuppressive therapy, among patients with LN, 10% to 30% of patients still progress to end-stage renal disease (ESRD), the ultimate manifestation of LN. Furthermore, up to 60% of the patients are unable to achieve the treatment targets with currently available therapeutic options.

The neonatal Fc receptor (FcRn) maintains constant levels of IgG in the serum by rescuing IgG antibodies from lysosomal degradation following uptake into cells. Given efgartigimod's mechanism of action of reducing IgG levels, efgartigimod may benefit patients with LN. Moreover, efgartigimod was shown to reduce circulating immune complexes in pemphigus patients (Example 1; FIG. 1). Since LN is primarily caused by renal deposition of immune complexes, this provides further proof-of-concept for treatment of LN and/or prevention of LN onset or progression by efgartigimod.

### A. Study Design

### Overall Design

This is a multicenter, randomized, double-blinded, placebo-controlled study to evaluate the efficacy and safety of efgartigimod IV in Chinese patients with active LN.

The study comprises a maximum 4-week screening period, a 24-week treatment period, and an 8-week follow-up period.

Approximately 60 participants who meet the eligibility criteria during the screening period are randomly assigned in a 1:1 ratio to receive weekly administration of efgartigimod IV or placebo, both in combination with standard of care (SoC; i.e., glucocorticoids and MMF/MPA) for 24 weeks. The randomization is stratified according to LN renal biopsy class (Class III/IV with V or Class III/IV without V).

The primary endpoint is change from baseline to week 24 in UPCR. Secondary endpoints include additional efficacy endpoints, PK, PD, immunogenicity, biomarkers, safety, and quality of life (see Table S6).

One week after receiving their last infusion of investigational medicinal product (IMP) (week 24), eligible participants have the option to roll over to an optional open-label follow-up study, with the goal of evaluating long-term safety of efgartigimod in this patient population. Participants who do not participate in the open-label follow-up study remain in the main study for an 8-week safety follow up.

A data safety monitoring board (DSMB) will review all unblinded safety data including the IgG levels during the study.

### Selection of Primary Endpoint

Substantial evidence has shown that an early reduction in proteinuria, particularly within 6 to 12 months from the start of treatment, is the single best predictor of improved long-term outcomes, including reduced risk of disease flares, ESRD, and death. Therefore, proteinuria, or UPCR, are useful parameters to evaluate effect of treatment and are preferred outcome measures by regulatory agencies.

Early reduction in UPCR indicates a long-term benefit in treatment of LN, which supports using it as the primary endpoint in this study.

### Rationale for Placebo Control

The design of this placebo-controlled study is acceptable for this patient population, given that SoC (immunosuppression with glucocorticoids and MMF/MPA) is administered in addition to placebo. A placebo-controlled study not including SoC in patients with LN would represent inadequate therapy for a population at risk for severe renal damage and potential ESRD.

### Justification for Dose

Based on results from prior clinical studies, including a phase 3 study in patients with generalized myasthenia gravis (gMG), administration of efgartigimod IV 10 mg/kg q7d for 4 infusions achieved near-maximal total IgG reduction, which resulted in a reduction of pathogenic autoantibodies, and was associated with clinical efficacy in patients with gMG. Furthermore, this dose was well tolerated with no safety concerns in all populations tested thus far.

LN patients are likely to have some degree of renal impairment and/or nephrotic proteinuria. Efgartigimod has a molecular weight of approximately 54 kDa and is therefore at the boundary of molecules that are renally filtered. After a single dose of efgartigimod IV 10 mg/kg in healthy subjects, less than 0.1% of the administered dose was recovered in urine. A population covariate analysis approach was used to evaluate the potential effect of the eGFR as a marker of renal function on the PK profile of efgartigimod. A statistically significant reduction in clearance with decreasing eGFR was identified, resulting in an increase of exposure. However, multiple IV doses up to 25 mg/kg are well tolerated in healthy volunteers. Therefore, a 10 mg/kg IV weekly dose was selected for this study to achieve maximum pharmacodynamic (IgG reduction) effects in this population.

### B. Study Population

Prospective approval of protocol deviations to recruitment and enrollment criteria, also known as protocol waivers or exemptions, is not permitted.

### Inclusion Criteria

Participants are eligible for study inclusion only if all of the following criteria apply:
Aged at least 18 years when signing the informed consent form;
Capable of giving signed informed consent, which includes compliance with the requirements and restrictions listed in the informed consent form and this protocol;
Diagnosis of SLE according to the Systemic Lupus Erythematosus International Collaborating Clinic (SLICC) 2012 classification criteria;
Active, proliferative LN Class III or IV [excluding Class III (C), IV-S (C), and IV-G (C)], either with or without the presence of Class V using the 2003 International Society of Nephrology (ISN)/Renal Pathology Society (RPS) criteria, proven by kidney biopsy within 6 months before randomization and confirmed by the central biopsy reading group;
Require LN induction therapy (glucocorticoids and MMF/MPA) based on investigator's clinical judgment. Induction therapy may begin before screening but should be initiated within 60 days before randomization;
Positive antinuclear antibody (ANA) test results defined by ANA titer ≥1:80 (based on Hep-2 immunofluorescence assay or equivalence by enzyme immunoassay) at screening;
Meet at least 1 of the following criteria at screening:
a. Elevated anti-dsDNA (≥30 IU/mL),
b. Low complement level (C3<0.9 g/L or C4<0.1 g/L);
Urine protein creatinine ratio (UPCR) ≥1.0 mg/mg at screening;
Estimated glomerular filtration rate (eGFR) ≥60 mL/min/1.73 m² at screening;
Serum total IgG ≥6 g/L at screening. Eligibility of participants with total IgG between 4 and 6 g/L at screening requires case-by-case discussion with the sponsor's medical monitor;
Contraceptive use by nonsterilized male participants and women of childbearing potential (WOCBP) consistent with local regulations, where available, for individuals participating in clinical studies. WOCBP must have a negative serum pregnancy test during screening and a negative urine pregnancy test at baseline to establish the nonpregnant state before receiving investigational medicinal product (IMP).

### Exclusion Criteria

Participants are excluded from the study if any of the following criteria apply:
Active or chronic infection requiring treatment:
a. Currently on any therapy for a chronic infection, including but not limited to tuberculosis, pneumocystis, cytomegalovirus, herpes simplex virus, herpes zoster, or atypical mycobacteria,
b. Active infection of any kind (excluding fungal infections of nail beds) or any major episode of infection requiring hospitalization or parenteral (IV or intramuscular) antibiotics treatment (e.g., antibacterials, antivirals, antifungals, or antiparasitic agents) within 60 days before randomization or oral antibiotics within 2 weeks before randomization;
Any evidence of central nervous system (CNS) lupus (including but not limited to seizures, psychosis, organic brain syndrome, cerebrovascular accident, cerebritis, or CNS vasculitis);
Currently on renal dialysis or expected to require dialysis during study period;
Previous kidney transplantation or planned transplantation during study period;
History of malignancy unless deemed cured by adequate treatment with no evidence of recurrence for ≥3 years before randomization. The following cancers are not exclusionary:
a. Adequately treated basal cell or squamous cell skin cancer,
b. Carcinoma in situ of the cervix,
c. Carcinoma in situ of the breast,
d. Incidental histological finding of prostate cancer (TNM stage TIa or TIb);
Heart failure (New York Heart Association [NYHA] Class III or IV) due to SLE, or any other severe cardiovascular involvement with safety concerns according to investigator's judgment;
Any other known autoimmune disease that, in the opinion of the investigator, would interfere with an accurate assessment of clinical symptoms of SLE/LN or put the participant at undue risk;
Clinical evidence of other significant or uncontrolled serious diseases (i.e., cardiovascular, pulmonary, hematologic, gastrointestinal, hepatic, renal, or neurological), have had a recent major surgery, or have any other condition, that in the opinion of the investigator, could confound the results of the study or put the participant at undue risk;
Previously failed treatment with MMF or any form of mycophenolate-based induction therapy, based on the investigator's opinion;
Systemic use of glucocorticoids within 12 months before randomization for chronic conditions excluding SLE (e.g., asthma, Crohn's disease);
Use of any monoclonal antibody within 3 months before randomization;
Previous treatment with plasmapheresis within 3 months before randomization;
Received CYC and/or CNIs within 2 months before randomization;
Received intravenous immunoglobulin (IVIg) within 4 weeks before randomization;
Use of complementary therapies, including Traditional Chinese Medicine, herbs, or procedure (e.g., acupuncture) within 4 weeks before randomization that can potentially interfere with the efficacy and safety of participants as assessed by the investigator;
Received live/live-attenuated vaccine within 28 days before randomization. The receipt of any inactivated, subunit, polysaccharide, or conjugate vaccine at any time before screening is not considered exclusionary. It is recommended that participants are up to date with vaccination(s) before the first dose of IMP;
Previously participated in a clinical study with efgartigimod;
Use of any investigational therapies within 3 months or 5 half-lives (whichever is longer) before randomization;
Positive serum test at screening for an active viral infection with any of the following conditions:
a. Hepatitis B virus (HBV) that is indicative of an acute or chronic infection unless associated with a negative HBV DNA test per the Centers for Disease Control and Prevention recommendation,
b. Hepatitis C virus (HCV) based on HCV antibody assay unless associated with a negative HCV RNA test,
c. HIV based on test results associated with either:
   (1) An AIDS-defining condition or a CD4 count <200 cells/mm³,
   (2) No adequate treatment with antiretroviral therapy;
SARS-CoV-2 antigen-based positive test at screening. The test is required regardless of whether the participant has been vaccinated;
Serum total IgG <4 g/L at screening;
Evidence of unstable kidney function defined as >20% decrease in eGFR within 3 months before randomization;
Has any of the following laboratory test values at screening:
a. Alanine aminotransferase (ALT) and/or aspartate aminotransferase (AST) >3× upper limit of normal (ULN),

$$Total bilirubin > 1.5 \times ULN , \qquad b .$$

$$Platelets < 75 \times {10}^{9} / L , \qquad c .$$

$$Neutrophils < 1.5 \times {10}^{9} / L , \qquad d .$$

$$Hemoglobin < 8 g / dL ; \qquad e .$$

Known hypersensitivity or contraindication to efgartigimod, any excipient of the IMP, or SoC medication used in the study;
In the opinion of the investigator, current or history of (i.e., within 12 months of randomization) alcohol, drug, or medication abuse;
Pregnant or lactating females and those who intend to become pregnant during study participation;
Any conditions or circumstances that in the opinion of the investigator make the participant unsuitable for the study.

### C. IMP(s) and Concomitant Therapy

Investigational medicinal product (IMP) is defined as any investigational intervention(s), marketed product(s), placebo, or medical device(s) intended to be administered to a study participant according to the study protocol.

### IMP(s) Administered

The IMP in this study includes efgartigimod IV and matching placebo (with the same formulation but without the active ingredient of efgartigimod), as described in Table S1:

TABLE S1
IMP(s) administered

| | | |
|---|---|---|
| Intervention Label | Efgartigimod IV | Placebo |
| Intervention Name | Efgartigimod IV | Placebo |
| Intervention | Sterile, colorless, clear | Sterile, colorless, clear |
| Description | concentrate solution for | concentrate solution for |
| | IV infusion efgartigimod | infusion, with the same |
| | 20 mg/mL, administered | excipients as |
| | IV | efgartigimod IV, but |
| | | without the active |
| | | ingredient (efgartigimod) |
| Type | Biologic | Placebo |
| Dose Formulation | Concentrate for solution | Concentrate for solution |
| | for IV infusion | for IV infusion |
| Unit Dose | 20 mg/mL | Not applicable |
| Strength(s) | | |
| Dosage Level(s) | 10 mg/kg weekly × 24 | Not applicable |
| | weeks | |
| Route of | IV infusion | IV infusion |
| Administration | | |
| Use | Experimental | Placebo-comparator |
| Sourcing | Provided centrally by the | Provided centrally by the |
| | sponsor | sponsor |
| Packaging and | IMP provided in glass | IMP provided in glass |
| Labeling | vials. Each vial labeled | vials. Each vial labeled |
| | as required per country | as required per country |
| | requirements | requirements |
| Former Name | ARGX-113 | Not applicable |

Any medication or vaccine (including over-the-counter or prescription medicines, vitamins, and/or herbal supplements) that the participant receives within 30 days of signing the informed consent form (ICF) or receives during the study participation must be recorded.

In addition, the participant's vaccination history within 12 months before signing the ICF, including any vaccination(s) for COVID-19, is recorded. The brand name of the vaccine and date of vaccination is also recorded, if known.

The sponsor's medical monitor should be contacted with any questions regarding concomitant or prior therapy.

All participants receive glucocorticoids as part of induction therapy for LN (see inclusion criterion). A combination of IV and oral glucocorticoids is permitted, and the initial IV pulse/bolus of methylprednisolone completed before randomization. The dosage of IV methylprednisolone for each participant will be determined based on the participant's clinical condition; the guideline-recommended dosage (250 mg to 500 mg/day for up to 3 days, allowing flexible dosing depending on the disease severity) is provided as a reference.

All participants must be receiving oral prednisone at an equivalent dose of ≥10 mg/day, but ≤60 mg/day (or 1.0 mg/kg/day, whichever is lower) at randomization. Glucocorticoid tapering regimens are specified by the protocol and are described below.

An initial oral prednisone dose of 0.5 to 1.0 mg/kg/day is recommended, not to exceed 60 mg/day, following intravenous methylprednisolone. The calculated dose is rounded to the nearest 10-mg increment listed in Table S2. The participant is instructed to initiate a glucocorticoid taper 2 weeks after the initiation of oral glucocorticoids according to the schedule listed in Table S2.

TABLE S2
Prednisone dose taper schedule

| Day | Starting prednisone dose taper schedule (mg/day) | | | | |
|---|---|---|---|---|---|
| Day 1 | 20 | 30 | 40 | 50 | 60 |
| Day 14 | 15 | 25 | 35 | 40 | 50 |
| Day 28 | 15 | 25 | 30 | 30 | 40 |
| Day 42 | 10 | 20 | 25 | 20 | 30 |
| Day 56 | 10 | 15 | 20 | 15 | 20 |
| Day 70 | 7.5 | 10 | 15 | 10 | 10 |
| Day 84 | 7.5 | 7.5 | 7.5 | 7.5 | 7.5 |

For LN flares, participants are optionally treated with prednisone (0.5 to 1.0 mg/kg/day, but ≤60 mg/day) for 2 weeks, followed by tapering to achieve ≤10 mg/day within 6 weeks after the initial glucocorticoid increase (see Table S3).

TABLE S3
Prednisone dose recommendations for LN flares

| Day | Starting prednisone dose taper schedule (mg/day) | | | | |
|---|---|---|---|---|---|
| Day 1 | 20 | 30 | 40 | 50 | 60 |
| Day 14 | 15 | 25 | 30 | 40 | 50 |
| Day 21 | 10 | 25 | 25 | 30 | 40 |
| Day 28 | 10 | 20 | 20 | 20 | 30 |
| Day 35 | 7.5 | 15 | 15 | 15 | 20 |
| Day 42 | 7.5 | 10 | 10 | 10 | 10 |

For extrarenal flares, based on the severity of the disease, participants are optionally retreated with prednisone up to 1.0 mg/kg/day (not to exceed 60 mg/day) and follow the recommended tapering schedule in Table S4. Intravenous glucocorticoids in equivalent doses are allowed if gastrointestinal involvement temporarily precludes oral glucocorticoids use.

TABLE S4
Prednisone dose recommendations for extrarenal flares

| Day | Starting prednisone dose taper schedule (mg/day) | | | | |
|---|---|---|---|---|---|
| Day 1 | 20 | 30 | 40 | 50 | 60 |
| Day 16 | 15 | 25 | 30 | 40 | 50 |
| Day 30 | 10 | 20 | 25 | 30 | 40 |
| Day 44 | 10 | 10 | 15 | 20 | 30 |
| Day 58 | 7.5 | 10 | 10 | 10 | 20 |
| Day 72 | 7.5 | 7.5 | 10 | 10 | 10 |
| Day 86 | 7.5 | 7.5 | 7.5 | 7.5 | 7.5 |

All participants either continue or initiate treatment with MMF (or MPA) during screening, as part of induction therapy for LN. MMF is titrated up to a maximum of 1.5 g to 2 g/day (or equivalent) in divided doses within 4 weeks.

Participants who started LN induction therapy ≥60 days before randomization are not eligible unless the previous induction was completed or terminated ≥2 months before randomization, and reinduction is required per the investigator's judgment.

For participants taking angiotensin-converting enzyme inhibitor (ACEi)/angiotensin receptor blocker (ARB), the dose of ACEi and/or ARB should be kept stable throughout the treatment period.

It is strongly recommended that all participants receive hydroxychloroquine (maximum dose of 5 mg/kg/day) at the screening/baseline visit and during the treatment period. The dose of hydroxychloroquine is kept stable unless a change is needed due to safety/tolerability concerns.

At the discretion of the investigator, bacterial prophylaxis is initiated at randomization or any time during the study in selected participants considered to be at high risk to develop infections.

At the discretion of the investigator, rescue therapy is indicated if renal function worsens (LN flare) during the treatment period as defined by any of the following:
Decreased eGFR by >20% (confirmed by repeated test) from baseline, accompanied by at least 1 of the following: 24-hour and/or spot UPCR of >1.0 mg/mg, red blood cell casts, or white blood cell casts; AND/OR
Increase in 24-hour UPCR:
   If the baseline UPCR is <3.5 mg/mg, an increase in 24-hour UPCR to ≥3.5 mg/mg AND increase in 24-hour UPCR greater than twice the value at baseline (confirmed by repeated test or spot UPCR with an interval within 2 weeks),
   If the baseline UPCR is ≥3.5 mg/mg, an increase in 24-hour UPCR greater than twice the value at baseline (confirmed by repeated test or spot UPCR with an interval within 2 weeks).

In these situations, the investigator can increase the glucocorticoid dose. Participants are treated with oral glucocorticoids with a prednisone dose of 0.5 to 1.0 mg/kg/day, but ≤60 mg/day for up to 2 weeks, with subsequent prednisone taper to achieve a prednisone dose of ≤10 mg/day within 6 weeks after starting taper. Further tapering is at the discretion of the investigator. Participants who do not exhibit a response to the initial 2-week course of increased glucocorticoids and then initiate a new immunosuppressive therapy (prohibited medication as defined in this study) are considered treatment failures.

If a participant is under or at immediate risk of developing severe forms of lupus clinical situations (including but not limited to lupus encephalopathy, pulmonary hemorrhage, and lupus crisis), the participant is discontinued from IMP or withdrawn from the study at any time and receives clinically appropriate individualized treatments at the investigator's discretion for the best interest of the participant.

Participants who experience an extrarenal SLE flare during the treatment period optionally receive rescue treatment with additional oral glucocorticoids. An extrarenal SLE flare is defined as an increase in the SLEDAI-2K score that is not accounted for by either hypocomplementemia, or an increase in anti-dsDNA antibody level, or the initiation of new immunosuppressive therapy, or an increase of corticosteroids if judged clinically appropriate by the investigator. The investigator should inform the sponsor's medical monitor about the clinical interventions for flare. These participants are optionally retreated with prednisone (up to 1.0 mg/kg/day, but ≤60 mg/day, based on the investigator's judgment) for up to 2 weeks on the basis of the severity of disease and organ system involvement; and then tapered to ≤10 mg/day following a separate tapering schedule. If participants require a new immunosuppressive therapy (other than glucocorticoids) for treatment of an extrarenal SLE flare, those participants are considered treatment failures.

Before initiation of rescue glucocorticoid therapy, all assessments are completed that pertain to the visit when the increased disease activity is identified, if possible. If the increased disease activity is identified between scheduled visits, then the participant returns to the clinic and all assessments listed for an unscheduled visit due to increased lupus activity are performed, including:
One 24-hour collection of urine for UPCR;
Spot UPCR;
SLEDAI-2K assessment;
Hematology, clinical chemistry, and urinalysis;
Vital signs and physical examination (optionally, a brief physical examination according to the participant's condition at the investigator's discretion);
Blood sample collection for PK, PD, and immunogenicity (predose, if applicable);
Blood sample collection for biomarker (predose, if applicable);
Other assessments by the investigator's judgment.

Participants who initiate rescue therapy continue IMP administration according to Table S5 or until the criteria for discontinuation of IMP are met.

Participants are allowed to receive corticosteroids for emergent illness (e.g., trauma, severe asthma) or surgery, if clinically warranted. Corticosteroids are given to participants at the discretion of the investigator to treat symptoms of adrenal insufficiency or steroid withdrawal. However, additional corticosteroid use in these scenarios should not exceed 2.5 mg/day for more than 7 days. In the event that surgery is required, the participant is instructed to inform related clinical personnel of their enrollment in a clinical study that does not permit the use of large doses of steroids. If possible, detailed surgical and anesthesia reports are provided to the investigator for review. These reports are kept in the participant's medical record to document the concomitant medications/therapies.

### Prohibited Medications

The following medications/treatments are prohibited:
Investigational therapies within 3 months or 5 half-lives (whichever is longer) before randomization and during the treatment period;
Any monoclonal antibody treatment within 3 months before randomization and during the treatment period;
CYC within 2 months before randomization and during the treatment period;
CNIs within 2 months before randomization and during the treatment period;
Complementary therapies including Traditional Chinese Medicine, herbs, or procedure (e.g., acupuncture) that potentially interferes with the efficacy and safety of participants as assessed by the investigator within 4 weeks before randomization and during the treatment period;
Live/live-attenuated vaccine within 28 days before randomization and during the treatment period;
Other immunosuppressive agents not specifically allowed in the study as described are not allowed during the study period.

### Restrictions of Concomitant Medications

The following are restrictions of concomitant medications:
A prescribed dose of MMF greater than 3 g/day, or a prescribed dose of MPA greater than 2160 mg/day at baseline and during the treatment period;
Initiation of ACEi/ARB treatment during the treatment period;
Initiation of antimalarial treatment during the treatment period;
IV glucocorticoids for LN during the treatment period;

### D. Study Assessments and Procedures

TABLE S5
Schedule of activities

| | Study Period | | | | | | | | | | | | | | | | | |
|---|---|---|---|---|---|---|---|---|---|---|---|---|---|---|---|---|---|---|
| | SCN | Treatment | | | | | | | | | | | | | | | | |
| | Visit | | | | | | | | | | | | | | | | | |
| | 1 | 2 | 3 | 4 | 5 | 6 | 7 | 8 | 9 | 10 | 11 | 12 | 13 | 14 | 15 | 16 | 17 | 18 |
| | Week | | | | | | | | | | | | | | | | | |
| | −4 to −1 | 0 | 0 | 1 | | 3 | 4 | 5 | 6 | 7 | 8 | 9 | 10 | 11 | 12 | 13 | 14 | 15 |
| | Day | | | | | | | | | | | | | | | | | |
| | −28 to −1 | 1 | 3 | 8 | 15 | 22 | 29 | 36 | 43 | 50 | 57 | 64 | 71 | 78 | 85 | 92 | 99 | 106 |
| | Window (day) | | | | | | | | | | | | | | | | | |
| | NA | NA | NA | ±3 days for each scheduled visit | | | | | | | | | | | | | | |
| Informed consent | X | | | | | | | | | | | | | | | | | |
| Eligibility criteria | X | X | | | | | | | | | | | | | | | | |
| Demography | X | | | | | | | | | | | | | | | | | |
| Medical history and | X | | | | | | | | | | | | | | | | | |
| current medical conditions | | | | | | | | | | | | | | | | | | |
| Kidney biopsy^{d} | X | | | | | | | | | | | | | | | | | |
| Total IgG, IgM, | X | | | | | | | | | | | | | | | | | |
| IgE, and IgA^{e} | | | | | | | | | | | | | | | | | | |
| Eligibility serology | X | | | | | | | | | | | | | | | | | |
| HIV/HBV/HCV^{f} | | | | | | | | | | | | | | | | | | |
| SARS-CoV-2 | X | | | | | | | | | | | | | | | | | |
| PCR test^{g} | | | | | | | | | | | | | | | | | | |
| Pregnancy test^{h} | X | X | | | | | X | | | | | | | | X | | | |
| FSH testⁱ | X | | | | | | | | | | | | | | | | | |
| Randomization | | X | | | | | | | | | | | | | | | | |
| EQ-5D-5L | | X | | | | | X | | | | | | | | X | | | |
| SLEDAI-2K | | X | | | | | X | | | | | | | | X | | | |
| assessment | | | | | | | | | | | | | | | | | | |
| CH50^{j} | | X | | | | | X | | | | | | | | X | | | |
| C3 and C4^{j} | X | X | | | | | X | | | | | | | | X | | | |
| Anti-dsDNA^{j} | X | X | | | | | X | | | | | | | | X | | | |
| ANA^{j} | X | X | | | | | X | | | | | | | | X | | | |
| C1q-binding circulating | X | X | | | | | X | | | | | | | | X | | | |
| immune complexes^{j} | | | | | | | | | | | | | | | | | | |
| 24-hr collection | X | | | | | | X | | | | | | | | X | | | |
| of urine for UPCR^{k} | | | | | | | | | | | | | | | | | | |
| 2 × 24-hr collection | | X | | | | | | | | | | | | | | | | |
| of urine for UPCR^{l} | | | | | | | | | | | | | | | | | | |
| Spot UPCR^{m} | | X | | | | | X | | | | | | | | X | | | |
| Hematologyⁿ | X | X | | | | | X | | | | | | | | X | | | |
| Clinical chemistry^{o} | X | X | | | | | X | | | | | | | | X | | | |
| Urinalysis^{p} | X | X | | | | | X | | | | | | | | X | | | |
| Vital signs^{q} | X | X | X | X | X | X | X | X | X | X | X | X | X | X | X | X | X | X |
| Physical examination^{r} | X | X | | | | | X | | | | | | | | X | | | |
| 12-lead electro- | X | X | | | | | X | | | | | | | | X | | | |
| cardiogram | | | | | | | | | | | | | | | | | | |
| Blood sample PK^{s} | | X | X | | | | X | | | | | | | | X | | | |
| Blood sample PD^{t} | | X | | X | X | X | X | | | | X | | | | X | | | |
| Blood sample | X | X | | | X | | X | | | | X | | | | X | | | |
| immunogenicity^{u} | | | | | | | | | | | | | | | | | | |
| Blood sample | | X | | | | | X | | | | | | | | X | | | |
| biomarkers^{v} | | | | | | | | | | | | | | | | | | |
| Weight (BMI at V2)^{w} | | X | | X | X | X | X | X | X | X | X | X | X | X | X | X | X | X |
| IMP^{x} | | X | | X | X | X | X | X | X | X | X | X | X | X | X | X | X | X |

| | |
|---|---|
| Standard of care | Continuous monitoring |
| Adverse event review | Continuous monitoring |
| Concomitant | Continuous monitoring |
| therapy review | |

| | Study Period | | | | | | | | | | | | |
|---|---|---|---|---|---|---|---|---|---|---|---|---|---|
| | Treatment | | | | | | | | | Follow-up^{a, b} | | NA | NA |
| | Visit | | | | | | | | | | | | |
| | 19 | 20 | 21 | 22 | 23 | 24 | 25 | 26 | 27 | 28 | 29 | UNS^{c} | ED^{d} |
| | Week | | | | | | | | | | | | |
| | 16 | 17 | 18 | 19 | 20 | 21 | 22 | 23 | 24 | 28 | 32 | NA | NA |
| | Day | | | | | | | | | | | | |
| | 113 | 120 | 127 | 134 | 141 | 148 | 155 | 162 | 169 | 197 | 225 | NA | NA |
| | Window (day) | | | | | | | | | | | | |
| | ±3 days for each scheduled visit | | | | | | | | | | | NA | NA |
| Informed consent | | | | | | | | | | | | | |
| Eligibility criteria | | | | | | | | | | | | | |
| Demography | | | | | | | | | | | | | |
| Medical history and | | | | | | | | | | | | | |
| current medical conditions | | | | | | | | | | | | | |
| Kidney biopsy^{d} | | | | | | | | | | | | | |
| Total IgG, IgM, | | | | | | | | | | | | | |
| IgE, and IgA^{e} | | | | | | | | | | | | | |
| Eligibility serology | | | | | | | | | | | | | |
| HIV/HBV/HCV^{f} | | | | | | | | | | | | | |
| SARS-CoV-2 | | | | | | | | | | | | (X) | |
| PCR test^{g} | | | | | | | | | | | | | |
| Pregnancy test^{h} | | | | | | | | | X | X | X | (X) | X |
| FSH testⁱ | | | | | | | | | | | | | |
| Randomization | | | | | | | | | | | | | |
| EQ-5D-5L | | | | | | | | | X | | | (X) | |
| SLEDAI-2K | | | | | | | | | X | | | (X) | |
| assessment | | | | | | | | | | | | | |
| CH50^{j} | | | | | | | | | X | X | X | (X) | X |
| C3 and C4^{j} | | | | | | | | | X | X | X | (X) | X |
| Anti-dsDNA^{j} | | | | | | | | | X | X | X | (X) | X |
| ANA^{j} | | | | | | | | | X | X | X | (X) | X |
| C1q-binding circulating | | | | | | | | | X | X | X | (X) | X |
| immune complexes^{j} | | | | | | | | | | | | | |
| 24-hr collection | | | | | | | | | | | | (X) | X |
| of urine for UPCR^{k} | | | | | | | | | | | | | |
| 2 × 24-hr collection | | | | | | | | | X | | | (X) | |
| of urine for UPCR^{l} | | | | | | | | | | | | | |
| Spot UPCR^{m} | | | | | | | | | X | | | (X) | X |
| Hematologyⁿ | | | | | | | | | X | | X | (X) | X |
| Clinical chemistry^{o} | | | | | | | | | X | | X | (X) | X |
| Urinalysis^{p} | | | | | | | | | X | | X | (X) | X |
| Vital signs^{q} | X | X | X | X | X | X | X | X | X | X | X | (X) | X |
| Physical examination^{r} | | | | | | | | | X | | X | (X) | X |
| 12-lead electro- | | | | | | | | | X | | X | (X) | X |
| cardiogram | | | | | | | | | | | | | |
| Blood sample PK^{s} | | | | | | | | | X | X | X | (X) | X |
| Blood sample PD^{t} | X | | | | X | | | | X | X | X | (X) | X |
| Blood sample | X | | | | X | | | | X | X | X | (X) | X |
| immunogenicity^{u} | | | | | | | | | | | | | |
| Blood sample | | | | | | | | | X | X | X | (X) | X |
| biomarkers^{v} | | | | | | | | | | | | | |
| Weight (BMI at V2)^{w} | X | X | X | X | X | X | X | X | | | | (X) | |
| IMP^{x} | X | X | X | X | X | X | X | X | | | | | |

| | |
|---|---|
| Standard of care | Continuous monitoring |
| Adverse event review | Continuous monitoring |
| Concomitant | Continuous monitoring |
| therapy review | |

ADA = antidrug antibodies;
ANA = antinuclear antibody;
BMI = body mass index;
dsDNA = double-stranded DNA;
ED = early discontinuation;
EQ-5D-5L = EuroQoL 5-Dimension 5-Level (questionnaire);
FSH = follicle-stimulating hormone;
HBV = hepatitis B virus;
HCV = hepatitis C virus;
Ig = immunoglobulin;
IMP = investigational medicinal product(s);
LN = lupus nephritis;
NA = not applicable;
PCR = polymerase chain reaction;
PD = pharmacodynamic(s);
PK = pharmacokinetic(s);
SCN = screening;
SLEDAI = Systemic Lupus Erythematosus Disease Activity Index;
UNS = unscheduled;
UPCR = urine protein creatinine ratio;
V = visit;
WOCBP = women of childbearing potential
Note:
When assessments coincide with IMP administration on the same visit day, examinations and sample collections (except for certain PK samples) are completed before dosing.
Note:
(X) = visit activities that are optional or only required under specific circumstances.
^{a}One week after receiving their final IMP dose (week 24, V27), eligible participants have the option to roll over to an open-label follow-up study in which they receive efgartigimod. Participants who do not participate in the open-label follow-up study remain in the main study for an 8-week safety follow-up.
^{b}For participants who received at least 1 dose of IMP and withdraw from the study (except for withdrawal of consent), the assessments listed for the ED visit are completed within 1 week after receiving their final IMP dose. A safety follow-up visit (as V29) 9 weeks (±3 days) after receiving their final IMP dose is also conducted.
^{c}In case of increased disease activity or safety reasons, the participant could come to the study site for an UNS visit.
^{d}A kidney biopsy within 6 months before randomization is required. The LN diagnosis and class must be confirmed by the central biopsy reading group.
^{e}Screening tests are performed at a local or a central laboratory.
^{f}Testing is performed at a local or a central laboratory. Results obtained within 28 days before screening are acceptable.
^{g}A positive PCR test for SARS-CoV-2 at screening is exclusionary for study participation. Nasal and/or throat mucosal cell samples are collected using site guidelines. Samples are tested for SARS-CoV-2 according to national and/or site level regulations and/or requirements.
^{h}WOCBP receive a serum pregnancy test at screening. Either serum or urine pregnancy tests are used at other visits. Screening and V2 pregnancy test results must be available before the participant is randomized.
ⁱFSH levels in the postmenopausal range are used to confirm a postmenopausal state in women; for postmenopausal women on hormonal therapy, the postmenopausal state is assessed by the investigator. Testing is performed at a local or a central laboratory.
^{j}CH50, C3, C4, anti-dsDNA, ANA, and C1q-binding circulating immune complexes are analyzed at a central laboratory and evaluated as biomarkers.
^{k}Participants collect 1 × 24-hour urine sample and give it to site staff at the clinic visit. Sample collection must begin within 2 days before each visit for V7, V15, and ED visit (if applicable). Analysis of the urine samples is performed at a central laboratory.
^{l}Participants collect 2 consecutive (separated by 7 to 14 days) 24-hour urine samples for V2 and V27 and give them to site staff at the clinic visit. The second sample collection must begin within 2 days before each visit. Analysis of the urine samples is performed at a central laboratory.
^{m}Participants provide a urine sample for a spot UPCR test. Analysis of the urine samples is performed at a central laboratory.
ⁿHematology parameters include RBC count, platelet count, hemoglobin, and hematocrit. RBC indices obtained for MCV, MCH, and MCHC. WBC count with differential (% and absolute numbers) obtained for neutrophils, eosinophils, lymphocytes, basophils, and monocytes. Testing is performed at a local or a central laboratory. For V2, results obtained within 7 days before V2 are acceptable.
^{o}Samples are collected after at least 8 hours of fasting (no food or drink, except water). Clinical chemistry parameters include ALT, AST, ALP, lactate dehydrogenase, GGT, total protein albumin, uric acid, blood urea/BUN, total cholesterol, HDL/HDL-C, LDL/LDL-C, triglycerides, total and direct bilirubin, glucose, creatinine, creatinine kinase, potassium, sodium, calcium, and chloride. Testing for screening is performed at a local or a central laboratory; testing for other scheduled visits and ED visit (if applicable) are performed at a central laboratory. For V2, a central laboratory result obtained within 7 days before V2 is acceptable.
^{p}Urinalysis parameters include specific gravity, pH, glucose, blood, ketones, bilirubin, urobilinogen, nitrite, leukocyte, urinary casts, and microscopic examination for RBC count and WBC count. Screening tests are performed at a local or a central laboratory; testing for other scheduled visits and ED visit (if applicable) is performed at a central laboratory. For V2, a central laboratory result obtained within 7 days before V2 is acceptable.
^{q}Vital sign parameters include blood pressure (systolic and diastolic pressure), pulse rate, respiratory rate, and body temperature. Participants must rest for at least 5 minutes in a quiet setting before the measurements are taken. Blood pressure and pulse rate are measured in a seated position. During visits when IMP is administered, vital signs are measured before and after the IMP infusion.
^{r}Complete physical examination includes assessments of the following: general appearance, respiratory, cardiovascular, abdomen, skin, head and neck (including ears, eyes, nose, and throat), lymph nodes, thyroid, musculoskeletal (including spine and extremities), and neurological systems. Complete physical examinations are performed for screening, V2, and V27; for other visits, brief physical examinations are performed according to the participant's condition at the investigator's discretion.
^{s}PK sample collection on V2 is performed within 1 hour pre-dose, at end of infusion (EOI; ±5 min), and 4 hours after infusion (±30 min); and on V7 and V15 within 1 hour pre-dose and at EOI (±5 min).
^{t}PD parameters include total IgG levels.
^{u}Blood samples for determination of ADA against efgartigimod for immunogenicity evaluation are collected. The immunogenicity sample taken at screening is used for validation purposes only
^{v}Biomarkers include anti-dsDNA, ANA, aCL, anti-Sm, anti-C1q, C3, C4, CH50, and C1q-binding circulating immune complexes.
^{w}Body weight is measured with participants attired in light clothing and without shoes. Height is measured without wearing shoes at V2. During the treatment period, body weight is measured before each IMP administration. The dose is calculated based on baseline (V2, day 1) body weight of the participant, but will be recalculated with a significant change in body weight (>10% compared to baseline).
^{x}The first dose is administered on day 1. The IMP is administered weekly as an intravenous infusion over 1 hour. Participants are observed for at least 30 minutes after the end of the infusion for routine safety monitoring.

### Urine Protein Creatinine Ratio (UPCR)

UPCR (in mg/mg) is measured in 24-hour, 2×24-hour, and spot urine samples. See Table S5 for timing and instructions for urine sample collections. Participants are given instructions on the procedure for collecting the 24-hour urine samples at home. The parameters include:
proteinuria;
urine creatinine;
UPCR.

### SLEDAI-2K Assessment

The SLEDAI-2K instrument is used to evaluate the disease activity of SLE (see Table S5). The results of the following assessments are required for a complete SLEDAI-2K evaluation:
Proteinuria (based on 24-hour collection of urine);
Platelets and white blood cells;
Urinary casts, microscopic examination for red blood cell count and white blood cell count in urine;
Body temperature;
Anti-dsDNA, C3, C4, and CH50.

### Circulating Immune Complex (CIC) Assessment

Blood samples are collected according to the schedule described in Table S5 and as detailed in the laboratory manual provided separately to study sites. CIC levels are determined using a validated method.

### Definitions of Clinical Outcomes

A complete renal response (CRR) is defined as:
eGFR ≥60 mL/min/1.73 m² or no confirmed decrease from the baseline eGFR value of >20%,

### AND

UPCR ≤0.5 mg/mg,

### AND

Did not fulfill the criteria of treatment failure.

A modified complete renal response (mCRR) is defined as:
eGFR ≥90 mL/min/1.73 m² or no confirmed decrease from the baseline eGFR value of >10%,

### AND

UPCR ≤0.5 mg/mg,

### AND

Did not fulfill the criteria of treatment failure.

A partial renal response (PRR) is defined as:
eGFR ≥60 mL/min/1.73 m² or no confirmed decrease from the baseline eGFR value of >20%,

### AND

≥50% decrease in UPCR from baseline, with 1 of the following conditions met:
   If the baseline UPCR is ≤3.0 mg/mg, then a UPCR of <1.0 mg/mg,
   If the baseline UPCR is >3.0 mg/mg, then a UPCR of <3.0 mg/mg,

### AND

Did not fulfill the criteria of treatment failure.

Treatment failure is defined as:
By week 24, the dose of glucocorticoids is >10 mg of prednisone/day,

### OR

Received any of the following prohibited medication or restricted concomitant medications during treatment period:
   A prescribed dose of MMF greater than 3 g/day, or a prescribed dose of MPA greater than 2160 mg/day,
   Initiate a new immunosuppressive therapy for LN, including but not limited to CYC, CNIs, rituximab, etc.,
   IV glucocorticoids for LN.

### E. Pharmacokinetics

Blood samples for PK analysis are collected at the time points described in Table S5. Efgartigimod serum concentrations are determined using a validated method.

### F. Pharmacodynamics

Blood samples for determination of total IgG levels in serum for PD evaluation are collected at the time points described in Table S5. Total IgG levels are determined using a validated method.

### G. Biomarkers

Blood samples are collected for biomarker research according to the schedule described in Table S5 and as detailed in the laboratory manual provided separately to study sites.

Biomarkers include:
Autoantibodies (anti-dsDNA, ANA, aCL, anti-Sm, and anti-C1q),
Complement (C3, C4, CH50, and C1q-binding circulating immune complexes).

### H. Immunogenicity Assessments

Blood samples are collected at the time points described in Table S5 to evaluate the serum levels of ADA against efgartigimod.

Samples are analyzed by the designated laboratory in a tiered approach using validated immunogenicity assays. Initially, samples are screened for a positive assay response (tier 1). Screened positive samples are then tested in a confirmation assay (tier 2). Finally, a titration of the ADA response is performed in positive tier 2 samples to characterize the magnitude of the antibody response (tier 3).

### I. Health Economics or Medical Resource Utilization and Health Economics

Participants complete the health-related quality of life questionnaire (EQ-5D-5L) at the scheduled time points indicated Table S5.

The EQ-5D-5L questionnaire is a standardized test recognized by many health authorities as a generic measure of health status for clinical and economic appraisal. The descriptive system comprises 5 dimensions: mobility, self-care, usual activities, pain/discomfort, and anxiety/depression. Scores for each dimension include 5 levels: no problem, slight problem, moderate problem, severe problem, extreme problem.

A visual analog scale (VAS) is included in the EQ-5D-5L. Participants mark their health status from 0 (the worst health you can imagine) to 100 (the best health you can imagine).

### J. Objectives and Endpoints

TABLE S6
Objectives and endpoints

| | |
|---|---|
| Objectives | Endpoints |

Primary

| | |
|---|---|
| Evaluate the clinical efficacy of | Change from baseline to week 24 in |
| efgartigimod IV compared to | UPCR |
| placebo in Chinese participants | |
| with LN as an add-on therapy to | |
| SoC | |

Secondary

| | |
|---|---|
| Further evaluate the efficacy of | Proportion of participants achieving |
| efgartigimod IV as assessed by | CRR at week 24 |
| additional endpoints | Time to CRR |
| | Proportion of participants achieving |
| | PRR at week 24 |
| | Time to PRR |
| | Change from baseline to week 24 in |
| | eGFR |
| | Change from baseline to week 24 in |
| | SLEDAI-2K score |
| | Change from baseline to week 24 in |
| | the dosage of glucocorticoids |
| | Treatment failure rate during |
| | treatment period |
| | Proportion of participants achieving |
| | mCRR at week 24 |
| Assess the PK of efgartigimod in | Efgartigimod serum |
| Chinese participants with LN | concentration-time profile |
| Assess the PD effect of | Absolute values, median changes |
| efgartigimod in Chinese | from baseline over time, and percent |
| participants with LN | changes from baseline in levels of |
| | total IgG |
| Assess the immunogenicity of | Incidence and prevalence of ADA |
| efgartigimod in Chinese | against efgartigimod |
| participants with LN | |
| Evaluate the effect of efgartigimod | Median changes from baseline over |
| on biomarkers in Chinese | time in biomarkers including |
| participants with LN | anti-dsDNA, ANA, aCL, anti-Sm, |
| | anti-C1q, C3, C4, CH50, and |
| | C1q-binding circulating immune |
| | complexes |
| Assess the safety of efgartigimod | Adverse events (AEs), including |
| IV in Chinese participants with LN | serious AEs (SAEs) and AEs of |
| | special interest (AESIs) |
| | Laboratory assessments, vital signs, |
| | and electrocardiogram (ECG) |
| Investigate the impact on quality | Change from baseline to week 24 in |
| of life of efgartigimod IV | EQ-5D-5L score |
| treatment in Chinese participants | |
| with LN | |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. A method of treating lupus nephritis in a subject in need thereof, the method comprising administering to the subject an effective amount of a human neonatal Fc receptor (FcRn) antagonist, wherein the FcRn antagonist comprises or consists of a variant Fc region or FcRn binding fragment thereof.

2-5. (canceled)

6. The method of claim 1, wherein the variant Fc region comprises or consists of a first Fc domain and a second Fc domain which form a homodimer or heterodimer, wherein the first Fc domain and/or the second Fc domain comprise amino acids Y, T, E, K, and F at EU positions 252, 254, 256, 433, and 434, respectively.

7. (canceled)

8. The method of claim 6, wherein the first Fc domain and/or the second Fc domain comprise amino acids Y, T, E, K, F, and Y at EU positions 252, 254, 256, 433, 434, and 436, respectively.

9. The method of claim 6, wherein the first Fc domain and/or the second Fc domain comprise an amino acid sequence independently selected from the group consisting of SEQ ID NO: 1, SEQ ID NO: 2, SEQ ID NO: 3, and SEQ ID NO: 30.

10. (canceled)

11. The method of claim 1, wherein the FcRn antagonist is efgartigimod.

12-13. (canceled)

14. The method of claim 1, wherein the FcRn antagonist is administered intravenously at a dose of 10 mg/kg to 30 mg/kg once weekly or once every two weeks.

15. The method of claim 1, wherein the FcRn antagonist is administered intravenously at a dose of 10 mg/kg once weekly.

16. The method of claim 1, wherein the FcRn antagonist is administered subcutaneously at a fixed dose of 750 mg to 3000 mg once weekly, once every two weeks, once every three weeks, once every four weeks, or once monthly.

17-22. (canceled)

23. The method of claim 1, further comprising administering to the subject an effective amount of a glucocorticoid, mycophenolate mofetil (MMF), mycophenolic acid (MPA), an angiotensin-converting enzyme inhibitor (ACEi), an angiotensin receptor blocker (ARB), hydroxychloroquine, a B-lymphocyte targeting agent, or any combination thereof.

24. The method of claim 23, wherein the glucocorticoid is methylprednisolone or prednisone.

25-37. (canceled)

38. A method of treating lupus nephritis in a subject in need thereof, the method comprising administering to the subject an effective amount of an FcRn antagonist within 60 days of the subject receiving an induction therapy for lupus nephritis.

39. The method of claim 38, wherein the induction therapy comprises administering to the subject an effective amount of a glucocorticoid.

40. The method of claim 38, wherein the induction therapy comprises administering to the subject an effective amount of methylprednisolone.

41. The method of claim 40, wherein the methylprednisolone is administered intravenously at a dose of 250 mg to 500 mg once a day for 1 to 3 days.

42-43. (canceled)

44. The method of claim 38, wherein the induction therapy comprises administering prednisone to the subject.

45. The method of claim 44, wherein the prednisone is administered orally at a dose of 10 mg/day to 60 mg/day.

46. (canceled)

47. The method of claim 44, wherein when the subject is administered the FcRn antagonist, the dose of prednisone is tapered over 12 weeks to a dose of 7.5 mg/day.

48. The method of claim 39, wherein the induction therapy further comprises administering to the subject a daily dose of an effective amount of MMF or MPA.

49. The method of claim 48, wherein the daily dose of MMF or MPA is up-titrated to 1.5 g/day to 2 g/day in divided doses within 4 weeks.

50. The method of claim 39, wherein the induction therapy further comprises administering to the subject a dose of an effective amount of hydroxychloroquine or a B-lymphocyte targeting agent.

51-55. (canceled)

56. The method of claim 1, wherein after administering the FcRn antagonist to the subject, the subject exhibits a post-administration estimated glomerular filtration rate (eGFR) of at least 60 mL/min/1.73 m² or that is less than 20% reduced as compared to a baseline eGFR obtained from the subject prior to administering the FcRn antagonist.

57-58. (canceled)

59. The method of claim 1, wherein after administering the FcRn antagonist to the subject, the subject exhibits a post-administration estimated glomerular filtration rate (eGFR) of at least 90 mL/min/1.73 m² or that is less than 10% reduced as compared to a baseline eGFR obtained from the subject prior to administering the FcRn antagonist.

60-61. (canceled)

62. The method of claim 1, wherein after administering the FcRn antagonist to the subject, the subject exhibits a post-administration urine protein creatinine ratio (UPCR) of at most 0.5 mg/mg.

63. The method of claim 1, wherein after administering the FcRn antagonist to the subject, the subject exhibits a post-administration UPCR that is at least 50% reduced as compared to a baseline UPCR obtained from the subject prior to administering the FcRn antagonist, and wherein if the baseline UPCR is at most 3 mg/mg, then the post-administration UPCR is less than 1 mg/mg or if the baseline UPCR is greater than 3 mg/mg, then the post-administration UPCR is less than 3 mg/mg.

64-65. (canceled)

66. The method of claim 1, wherein after administering the FcRn antagonist to the subject, the subject exhibits a post-administration Systemic Lupus Erythematosus Disease Activity Index (SLEDAI)-2K score that is reduced as compared to a baseline SLEDAI-2K score obtained from the subject prior to administering the FcRn antagonist.

67. (canceled)

68. The method of claim 1, wherein after administering the FcRn antagonist to the subject, the subject exhibits a post-administration level of a serum autoantibody that is reduced as compared to a baseline level of the serum autoantibody obtained from the subject prior to administering the FcRn antagonist.

69-71. (canceled)

72. The method of claim 1, wherein after administering the FcRn antagonist to the subject, the subject exhibits a post-administration level of a serum complement that is reduced as compared to a baseline level of the serum complement obtained from the subject prior to administering the FcRn antagonist.

73-89. (canceled)


SEQUENCE LISTING (33 sequences)

SEQ ID NO: 1 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 216 aa, synthetic construct
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 5 - protein, 445 aa, synthetic construct
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 219 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 214 aa, synthetic construct
DIQMTQSPSSLSASVGDRVTITCKASDHINNWLAWYQQKPGQAPRLLISGATSLETGVPSRFSGSGTGKDYTLTISSLQPEDFATYYCQQYWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 444 aa, synthetic construct
QVQLVQSGAELKKPGASVKLSCKASGYTFTSYGISWVKQATGQGLEWIGEIYPRSGNTYYNEKFKGRATLTADKSTSTAYMELRSLRSEDSAVYFCARSTTVRPPGIWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 15 aa, synthetic construct
EPKSCDKTHTCPPCP

SEQ ID NO: 13 - protein, 227 aa, synthetic construct
  VARIANT 1: note = D or absent
  VARIANT 2: note = K or absent
  VARIANT 3: note = T or absent
  VARIANT 4: note = H or absent
  VARIANT 5: note = T or absent
  VARIANT 6: note = C or absent
  VARIANT 226: note = G or absent
  VARIANT 227: note = K or absent
XXXXXXPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPXX

SEQ ID NO: 14 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 15 - protein, 226 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 16 - protein, 225 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 17 - protein, 224 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 18 - protein, 225 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 19 - protein, 224 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 20 - protein, 223 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 21 - protein, 223 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 22 - protein, 222 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 23 - protein, 221 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 24 - protein, 221 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 25 - protein, 220 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 26 - protein, 219 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 27 - protein, 224 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 28 - protein, 223 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 29 - protein, 222 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 30 - protein, 222 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 31 - protein, 220 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 32 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 33 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCPNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY